(19) United States
(12) Patent Application Publication
(10) Pub. No.: US 2023/0107824 A1
(43) Pub. Date: Apr. 6, 2023

(54) MODIFIED GENOMES AND USE THEREOF

(71) Applicant: SYNVACCINE LTD., Tel Aviv (IL)

(72) Inventors: Tamir TULLER, Tel Aviv (IL); Yoram ZARAI, Tel Aviv (IL); Modi ROOPIN, Gedera (IL); Zohar ZAFRIR, Tel Aviv (IL)

(21) Appl. No.: 17/961,274

(22) Filed: Oct. 6, 2022

Related U.S. Application Data
(63) Continuation of application No. PCT/IL2021/050412, filed on Apr. 11, 2021.
(60) Provisional application No. 63/008,050, filed on Apr. 10, 2020.

(51) Int. Cl. A61K 39/12; G16B 25/10; G16B 20/00; C12N 7/00

(52) U.S. Cl. CPC A61K 39/12; G16B 25/10; G16B 20/00; C12N 7/00; C12N 2770/24121; C12N 2770/24162

(57) ABSTRACT

Modified genomes of an organism comprising at least one coding sequence comprising at least one mutation, wherein the mutation generates an underrepresented sequence that is underrepresented in the unmodified genome of the organism are provided. Organism and cells comprising the modified genomes of the invention, pharmaceutical compositions comprising the organisms and cells of the invention, as well as methods of making the modified genomes are also provided.

### CROSS REFERENCE TO RELATED APPLICATIONS

This application claims the benefit of priority of U.S. Provisional Patent Application No. 63/008,050, filed Apr. 10, 2020, the contents of which are all incorporated herein by reference in their entirety.

### REFERENCE TO AN ELECTRONIC SEQUENCE LISTING

The contents of the electronic sequence listing (SYV-P-006-US.xml; Size: 10,087 bytes; and Date of Creation: Oct. 6, 2022) is herein incorporated by reference in its entirety.

### FIELD OF INVENTION

The present invention is in the field of virus attenuation and vaccine production.

### BACKGROUND OF THE INVENTION

Viruses, the most abundant type of biological entity, are small infectious agents that can only replicate inside the living cells of other organisms (hosts). The viral genetic material is composed of either RNA or DNA molecule, single or double stranded. Viral genomes typically encode three types of protein: proteins for replicating the genome, proteins for packing the genome, and proteins for modifying the function of the host's cell in order to enhance the replication of the virus's material.

Viruses are believed to play a central role in evolution, (e.g., via horizontal gene transfer (HGT)), be responsible for various human diseases (e.g., AIDS and respiratory diseases), and also have important applications to biotechnology and nanotechnology. For instance, the recent Zika virus epidemic in the Americas, and the novel coronavirus (2019-nCoV) outbreak in China have led the World Health Organization to declare a “public health emergency of international concern”. Due to their complete reliance on the host gene expression machinery, viruses are under constant evolutionary pressure to effectively interact with the host intracellular factors, and at the same time effectively evade its immune system. Thus, understanding how viruses co-evolve with their hosts in order to ensure their fitness may help in developing novel viral based applications such as vaccines, oncologic therapies, and anti-bacterial treatments.

It is natural to expect that viruses and hosts co-evolution patterns are also encrypted in the viral genome. For example, it was shown that high correlation of GC content exists between bacteriophage and related hosts, that a pattern of CpG dinucleotides is suppressed in vertebrate hosts and related RNA viruses, that the frequency of TpA dinucleotides is suppressed in invertebrate hosts and related RNA viruses, and that many long sequences are shared between hosts and their related viruses.

Identification and analysis of short DNA sequences that are under-represented (also referred to as suppressed or avoided) in genomes of different species were analyzed in the past. These studies have focused on palindromic repeats and targets of the bacterial endonuclease system. An analysis of under-represented nucleotide sequences in the coding regions of all types of viruses and in the coding regions of their corresponding hosts has not yet been carried out.

Effective manufacture of vaccines remains an unpredictable undertaking. There are three major kinds of vaccines: subunit vaccines, inactivated (killed) vaccines, and attenuated live vaccines. For a subunit vaccine, one or several proteins from the virus (e.g., a capsid protein made using recombinant DNA technology) are used as the vaccine. Subunit vaccines produced in Escherichia coli or yeast are very safe and pose no threat of viral disease. Their efficacy, however, can be low because not all of the immunogenic viral proteins are present, and those that are present may not exist in their native conformations.

Inactivated (killed) vaccines are made by growing more-or-less wild type (WT) virus and then inactivating it, for instance, with formaldehyde (as in the Salk polio vaccine). A great deal of experimentation is required to find an inactivation treatment that kills the entire virus and yet does not damage the immunogenicity of the particle. In addition, residual safety issues remain in that the facility for growing the virus may allow a virulent virus to escape or the inactivation may fail.

An attenuated live vaccine comprises a virus that has been subjected to mutations rendering it to a less virulent and usable for immunization. Live, attenuated viruses have many advantages as vaccines: they are often easy, fast, and cheap to manufacture; they are often easy to administer (the Sabin polio vaccine, for instance, was administered orally on sugar cubes); and sometimes the residual growth of the attenuated virus allows “herd” immunization (immunization of people in close contact with the primary patient). These advantages are particularly important in an emergency, when a vaccine is rapidly needed. The major drawback of an attenuated vaccine is that it has some significant frequency/probability of reversion to WT virulence. For example, for this reason, the Sabin vaccine is no longer used in the United States.

To overcome the numerous pitfalls attributed to the classical vaccine design strategies, more efficient and robust rational approaches based on computer-based methods are highly desirable. One direction in designing in-silico vaccine candidates may be based on exploiting the synonymous information encoded in the genomes for attenuating the viral replication cycle while retaining the wild type proteins.

Some existing computational strategies may propose methods for designing life attenuated viral strains by using the additional layer of information carried by the distribution of codons encoding the viral proteome. However, these have been tested only on a limited variety of viruses, were based on specific global features encoded in the genomes (while ignoring other important, possibly local, factors), and did not take into consideration the evolutionary dynamics as a general determinant of a possible significance of various genomic features for the viral replication cycle.

Accordingly, there remains a need for a systematic approach to generating attenuated live viruses that have practically no possibility of reversion and thus provide a fast, efficient, and safe method of manufacturing a vaccine.

### SUMMARY OF THE INVENTION

The present invention provides modified genomes of an organism comprising at least one coding sequence comprising at least one mutation, wherein the mutation generates an underrepresented sequence that is underrepresented in the unmodified genome of the organism. Organisms and cells comprising the modified genomes of the invention, as well as methods of making the modified genomes are also provided.

According to a first aspect, there is provided a modified viral genome comprising at least one coding sequence comprising at least one mutation, wherein the mutation generates a sequence of 3, 4 or 5 nucleotides that is underrepresented in an unmodified genome of the virus.

According to another aspect, there is provided a method of generating a modified genome, the method comprising receiving a sequence of a genome of an organism, selecting at least one coding sequence within the genome sequence and introducing at least one mutation into the at least one coding sequence wherein the mutation generates a sequence of 3, 4 or 5 nucleotides that is underrepresented in an unmodified genome of the organism.

According to another aspect, there is provided a modified genome produced by a method of the invention.

According to another aspect, there is provided an attenuated virus comprising a modified genome of the invention.

According to another aspect, there is provided a vaccine composition comprising the attenuated virus of the invention.

According to another aspect, there is provided a computer program product for generating a modified genome, comprising a non-transitory computer-readable storage medium having program code embodied thereon, the program code executable by at least one hardware processor to:
a. receive a sequence of a genome of an organism;
b. receive a list of underrepresented sequences of length 3, 4 or 5 nucleotides in the organism;
c. calculate mutations within at least one coding sequence of the genome that generate at least one underrepresented sequence from the list; and
d. provide an output modified genome comprising the at least one coding sequence comprising at least one calculated mutation wherein the at least one calculated mutation does not alter an amino acid sequence of a protein encoded by the at least one coding sequence.

According to some embodiments, the at least one mutation is a synonymous mutation and does not alter an amino acid sequence encoded by the at least one coding sequence.

According to some embodiments, the underrepresented sequence is a homooligonucleotide sequence comprising 3, 4 or 5 of the same nucleotide base.

According to some embodiments, the underrepresented sequence is a palindromic sequence that is identical to its reverse complement.

According to some embodiments, the underrepresented sequence is neither a homooligonucleotide nor a palindromic sequence that is identical to its reverse complement.

According to some embodiments, the underrepresented sequence is underrepresented in all three reading frames of the unmodified genome.

According to some embodiments, the virus is an RNA virus, a single-stranded DNA virus or a double-stranded DNA virus.

According to some embodiments, the virus is a double-stranded DNA virus and the underrepresented sequence is a palindromic sequence that is identical to its reverse complement.

According to some embodiments, the underrepresented sequence is also underrepresented in a genome of a host organism that is infectable by the virus.

According to some embodiments, the host organism is a bacterium.

According to some embodiments, the host organism is a vertebrate.

According to some embodiments, the virus is ZIKA virus.

According to some embodiments, the underrepresented sequence is selected from TTT, AAA, TAG, CCC, GAC, GGG, AAAA, TTTT, GATC, CGCG, GGGG, CCCC, AAAAA, TTTTT, GGATC, GATCT, GGGGG, and CCCCC.

According to some embodiments, the virus is:
a. a double stranded DNA virus that infects bacteria and the underrepresented sequence is a palindromic sequence selected from TTAA, ATAT, CTAG, TGCA, ACGT, CATG, TATA, GTAC, AGCT, GCGC, TCGA, CCGG, AATT, GGCC, CGCG, and GATC;
b. a double stranded DNA virus that infects vertebrates and the underrepresented sequence is a palindromic sequence selected from TTAA, TGCA, TCGA, TATA, GTAC, GGCC, GCGC, GATC, CTAG, CGCG, CCGG, CATG, ATAT, AGCT, ACGT and AATT; or
c. a double stranded DNA virus that infects vertebrates and the underrepresented sequence is a palindromic sequence selected from GATC, GTAC, TGCA, ACGT, GGCC, TCGA, and AATT.

According to some embodiments, the mutated coding sequence encodes a protein identical to an unmutated coding sequence, and wherein a mutated coding sequence comprises every mutation that increases the number of underrepresented sequences without altering the encodes protein sequence.

According to some embodiments, the mutated coding sequence comprises at least 90 mutations.

According to some embodiments, the coding sequence encodes a protein that is not a surface protein.

According to some embodiments, the coding sequence encodes a surface protein and the underrepresented sequence is a sequence selected from ACT, CTT, GAC, CCT, AAA, AGG, AAT, AGT, TGA, ACC, GTC, TTC, TAG, GGG, GTG, CTAC, CCCC, ACCT, TTTT, CCCCC, TTGCC, and CTTGC.

According to some embodiments, the coding sequence encodes at least one of:
a. a structural protein and the underrepresented sequence is a sequence selected from TTT, AAA, GAG, GGG, GGA, CCC, GCT, GCC, CGA, GTC, AGG, CTC, TGT, GAC, AAT, TTTT, AAAA, GATC, GGGG, GGCT, AATT, CGCG, AGCT, GCTT, CCCC, GGAG, GTAC, AAAT, AGCC, TCAG, AAAAA, TTTTT, GGATC, GATCA, CCTGG, AAATT, CGCGC, TTTTC, AATTT, CTTCA, CCCCC, AGATC, and AGCTC;
b. an enzymatic protein and the underrepresented sequence is a sequence selected from TTT, AAA, GAG, GGA, CGC, TGT, GGG, AAT, GCT, TAG, AGG, CGA, GTC, GAC, CCC, AAAA, TTTT, GATC, CGCG, AATT, AGCT, GGCT, TCGA, GGGG, CCCC, GGCC, GCGC, GGAG, GTAC, TTGG, AAAAA, TTTTT, GATCT, AGATC, GGATC, GATCA, CCTGG, AAATT, AATTT, CCCCC, CGATC, TTCGA, CTTGG, TTTTC, and AGCTT;
c. a protein of unknown or uncharacterized function and the underrepresented sequence is a sequence selected from TTT, AAA, TAG, CCC, TGT, CGC, GAC, GGG, AAT, ACA, CTC, GAG, GTC, GCT, GGA, AAAA, TTTT, GATC, CCCC, GGCC, CGCG, GGGG, AATT, AGCT, CCGG, GGCT, GCGC, GTAC, AGCC, TCGA, AAAAA, TTTTT, AGATC, GATTCT, GATCA, GGATC, CCCCC, AATTT, CCTGG, GGGGG, AAAAT, AAATT, TGGCT, AGCTT, and ATTTT; and
d. a protein of known or characterized function that is not a surface, structural or enzymatic protein and the underrepresented sequence is a sequence selected from TTT, AAA, CCC, GGG, ACT, GAC, TAG, CTC, GGA, CGC, GCG, GTG, GAG, AGT, CGA, AAAA, GATC, TTTT, GGGG, CCCC, AATT, CGCG, GCGC, GGAG, AGCT, GGCT, TCGA, GTAC, GGCC, CTCC, AAAAA, CCCCC, GGGGG, TTTTT, GATCT, AGATC, GGATC, GATCA, CCTGG, CGCGG, CCCCA, AAAAT, CGCGC, GGAGC, and GGCGC.

According to some embodiments, the organism is a virus, optionally, wherein the virus is an RNA virus, a single-stranded DNA virus or a double-stranded DNA virus.

According to some embodiments, the underrepresented sequence is also underrepresented in a genome of a host organism that is infectable by the virus, optionally wherein the host organism is selected from a bacterium and a vertebrate.

According to some embodiments, the introducing comprises mutating every nucleotide that increases the number of underrepresented sequences within the coding sequence without altering an amino acid sequence of a protein encoded by the coding sequence.

According to some embodiments, the introducing comprises introducing at least 90 mutations into the at least one coding sequence.

According to some embodiments, the output modified genome comprises the at least one coding sequence comprising every possible calculated mutation that does not alter an amino acid sequence of a protein encoded by the at least one coding sequence.

According to some embodiments, the list is ranked in order of the extent of underrepresentation in the genome.

According to some embodiments, the output modified genome comprises the 5 most highly underrepresented sequences that could be generated by a synonymous mutation.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-C: Provided are the analysis flow diagram (1A), summary of the viruses-hosts association database (1B), where left values specify the total number of viruses corresponding to each host domain, and right values specify the total number of hosts in each host domain, and randomization models (1C), illustrating an example of dinucleotides randomization (left) and synonymous codons randomization (right).

FIG. 2: A general scheme of engineering a synthetic sequence. Specifically, in the case of the synthetic ZIKV UR99 sequence, we introduced different under-represented 5-mer oligos in the first two reading frames (identified using both randomization models), replacing the original nucleotide sequence while verifying that the protein amino acid sequence remains unchanged.

FIGS. 3A-B: Average number of under-represented sequences of size m=3, 4, and 5 nucleotides are provided. (3A) The average number in the original viral genome among different subsets of viruses. (3B) The average number in the random viral genome (i.e., in a random variant of the virus) among different subsets of viruses (see Materials and Methods). The virus's subsets are denoted by a pair V:H, indicating all viruses of type V that infect hosts of domain H (H defines the first two letters of the host domain). For example, ssRNA:Pl denotes all ssRNA viruses that infect hosts of domain plants.

FIGS. 4A-B: The most abundant common under-represented sequences of size m=3 (top panel in each sub-figure), 4 (middle panel in each sub-figure) and 5 (bottom panel in each sub-figure). (4A) In five host domains (left) (no common under-represented sequences were found for hosts of the fungi domain) and in the main five virus groups (right). (4B) In subsets of hosts (left) and subsets of viruses (right). The host subsets are denoted by the pair H:V, indicating all hosts of domain H that are infected by viruses of type V (H defines the first two letters of the host domain). For example, Ve:dsDNA denotes all hosts of the domain vertebrate that are infected by viruses of type dsDNA. The virus subsets are denoted by the pair V:H, indicating all viruses of type V that infect hosts of domain H. For example, ssRNA:Pl denotes all ssRNA viruses that infect hosts of domain plants. Each row in each panel denotes a nucleotide sequence. A maximum of 15 sequences are shown in each panel ordered top to bottom based on their occurrence frequency (i.e., top sequence appeared most frequently as common under-represented).

FIGS. 5A-F: Under-represented palindrome sequences. (5A) The percentage of palindromic sequences of size m=4 nucleotides that are common under-represented in hosts of domain bacteria that are infected by viruses of type dsDNA. (5B) The percentage of palindromic sequences of size m=4 nucleotides that are common under-represented in viruses of type dsDNA infecting hosts of domain vertebrate (left) and hosts of domain bacteria (right). (5C) The number of occurrences of each palindrome of size m=4 as under-represented sequence in viruses of type dsDNA infecting hosts of domain bacteria in the original viral genome (blue) and in the randomized genome (red) of viruses. Note that the scales of the blue and the red bars are extremely different. (5D) The number of occurrences of each palindrome of size m=4 as under-represented sequence in viruses of type dsDNA infecting hosts of domain vertebrate in the original viral genome (blue) and in the randomized genome (red) of viruses. Note that the scales of the blue and the red bars are extremely different. (5E) Overlap between common under-represented sequences of size m=4 nucleotides in dsDNA viruses and restriction sites downloaded from the REBASE database. Shown are the number of exact matches between the most abundant common under-represented palindromes of size m=4 in dsDNA viruses and restriction sites. The corresponding restriction enzyme names and p-values are shown as well. (5F) The number of restriction sites that are a superset of the most abundant common under-represented palindromes of size m=4 nucleotides in dsDNA viruses. Shown also are the corresponding p-values.

FIGS. 6A-C: The number of the common under-represented nucleotide sequences in subsets of hosts and in subsets of viruses. 6A, 6B, and 6C correspond to sequences of size m=3, 4, and 5 nucleotides, respectively, where in each panel the left sub figure corresponds to subsets of hosts and the right sub figure to subsets of viruses.

FIGS. 7A-C: The most abundant common under-represented nucleotide sequences that are shared between hosts and their corresponding viruses in different subsets of hosts and viruses. (7A) Class A sequences (left) of size m=3 (top panel), 4 (middle panel), and 5 (bottom panel) and unique class A sequences (right) of size m=4 (top panel) and 5 (bottom panel). (7B) Class B sequences (left) of size m=3 (top panel), 4 (middle panel), and 5 (bottom panel) and unique class B sequences (right) of size m=4 (top panel) and 5 (bottom panel). (7C) Class C sequences (left) of size m=3 (top panel), 4 (middle panel), and 5 (bottom panel) and unique class C sequences (right) of size m=4 (top panel) and 5 (bottom panel). Each row in each panel denotes a nucleotide sequence. A maximum of 15 sequences are shown in each panel ordered top to bottom based on their occurrence frequency (i.e., top sequence appeared most frequently as common under-represented).

FIGS. 8A-B: Under-represented sequences within the virus functional gene sets. Here, “surf” stands for surface, “strc” for structural, “enzm” for enzymatic, “unkn” for unknown (unclassified), and “othr” for other (hypothetical) functional groups. (8A) The average number of under-represented sequences, over all three reading frames, of size m=3, 4, and 5 nucleotides, identified in each viral gene set when analyzing (randomly selected) 1500, 1240, 1450, 3300 and 2210 genes from each of the surface, structural, enzymatic, unknown and hypothetical functional groups, respectively. (8B) The most abundant common under-represented nucleotide sequences in each of the virus functional group, of size m=3 (upper panel), m=4 (middle panel), and m=5 (lower panel). Each row in each panel denotes a nucleotide sequence. A maximum of 15 sequences are shown in each panel ordered top to bottom based on their occurrence frequency (i.e., top sequence appeared most frequently as common under-represented).

FIGS. 9A-D: Incorporation of under-represented sequences produced an attenuated ZIKV variant. (9A) Foci size and replication kinetics of WT ZIKV and UR99 in Vero cells. The smaller foci size comparison demonstrates variant attenuation of the UR99 (bottom right). Titer analysis shows the UR99 variant attenuation relative to WT ZIKV (borderline significant p-value in day two: 0.078). (9B) Mortality curves of AG129 mice infected with UR99, synthetic WT ZIKV or Malaysian strain ZIKV. (9C) Average weight change, in percentage, of animals infected with WT ZIKV Malaysian, synthetic WT ZIKV, or UR99. (9D) PRNT50 titers from serum collected from vaccinated AG129 mice 13 days post vaccination (****P<0.0001, **P<0.01 as compared with vehicle treatment).

### DETAILED DESCRIPTION OF THE INVENTION

The present invention, in some embodiments, provides a modified genome of an organism comprising at least one mutation that generates an underrepresented sequence in that organism. The present invention further concerns a method of producing a modified genome, and cells and organisms comprising the modified genomes. A computer program product for designing the modified genomes is also provided, as is a pharmaceutical composition comprising the cells and/or organisms of the invention.

The invention is based on the following surprising results. Herein we analyzed sequences of three, four and five nucleotides in length that are under-represented in the coding regions of viruses of all types and in their corresponding host coding regions. This analysis is based on a novel statistical evaluation that controls for classical coding region features, which is performed separately in each of the three reading frames. We provide various novel discoveries that may shed light on the evolution of viral DNA sequences, and on the virus co-evolution with its respective hosts. It is important to emphasize that the observed patterns may be related to various variables and their complex interactions, include gene expression optimizations, various mechanisms for escaping the host immune system, and co-evolution with the corresponding hosts.

In general, our analysis reveals that under-represented viral sequences are related to different mechanisms such as restriction modification systems, and possibly to alternative or unknown immune escape mechanisms, as these sequences cannot be explained by canonical mechanisms that may suggest, for example, classical viral recognition employing antibodies.

We show that homooligonucleotide repeats are the most abundant under-represented sequences in both viruses and hosts. A possible explanation for this avoidance is to reduce an erroneous ribosomal frame shifts and thus reduce faulty translation and consequentially the overall translation cost. However, as this motif is shown to be shared between hosts and viruses, our analysis also indicates that a stronger selection pressure against these sequences exists in viruses. This again can be attributed to escape mechanisms from the host immune system, as the virus nucleotide composition evolves to be similar to the host, and it is certainly possible that an excess avoidance of homooligonucleotide repeats reduces viral recognition by classical host immune mechanisms. There may be other relevant explanations such as interaction with small RNA genes (e.g., miRNAs). It is possible, for example, that these sequences may increase the efficiency of miRNA and mRNA interactions and thus decrease expression levels.

In addition to homooligonucleotide repeats, we show that palindromes are among the most abundant under-represented sequences in viruses. Specifically, excluding homooligonucleotide repeats, our analysis reveals that 51% of all under-represented sequences of four nucleotides long in viruses are palindromes (where only 6.25% of all possible sequences of that size are palindromes).

Indeed, analysis of palindromes avoidance in viruses was performed previously. It was shown that palindromes are the most under-represented short sequences in a prokaryotic genome. For example, it was reported that short palindromic sequences are avoided at a statistically significant level in the genomes of several bacteria. These analyses are based on statistical counts of certain sequences in the given DNA and thus do not control for canonical coding region features (codon usage bias, amino acid order and content and dinucleotide distribution) as was done in this study. In addition, our analysis is performed over a large set of viruses of all types and their corresponding hosts, and at a reading frame resolution. Thus, we believe that the results reported here may be more accurate and will provide better understanding of this phenomenon.

One plausible explanation for avoidance of palindromes in viruses is because they are targets for many restriction-modification systems and possibly for general recombination systems as well. We statistically show a high overlap between under-represented palindromes in viruses and restriction enzyme patterns. This overlap cannot be explained by classical coding region features. Restriction of recognition sites has been observed in genomes of prokaryotic organisms. It was also shown that the recognition site avoidance correlates with the lifespan of restriction-and-modification systems. The method employed previously is based on a compositional bias calculation, which is the ratio of the observed to the expected frequency of a sequence, where the expected frequency is estimated based on the observed frequencies of all subsites of a given sequence. Since the compositional bias measure doesn't account for a statistical background that preserves know evolutionary forces, we believe that a more accurate and comprehensive procedure of identifying under-represented sequences is the one employed here.

In addition, we analyze the distribution of these under-represented sequences among various viral and host groups. We show, for example, that dsDNA viruses infecting bacteria or vertebrate hosts contain a larger set of under-represented sequences than other viral types, and that this may be related to their larger genome size. Furthermore, we show that on average the set of sequences that are under-represented in viruses but are not under-represented in their related hosts is the largest set among different host-virus under-represented correspondence.

We also show that the selection against under-represented sequences in viruses depends upon the protein function. For example, larger number of sequences are shown to be under-represented in enzyme genes than in surface genes. Moreover, even larger number of sequences are found to be under-represented in genes with (currently) unknown functionality, prompting further investigation into the nature of these genes. The differences between these groups may also be related to the expression levels of the different proteins. If, for example, surface genes tend to have low expression levels then they may be under weaker selection for features such as under-represented sequences.

Vaccines are a topic of a singular importance in present day biomedical science. However, the discovery of vaccines has so far been primarily empirical in nature requiring considerable investments of time, efforts, and resources. To overcome the numerous pitfalls attributed to the classical vaccine design strategies, more efficient and robust rational approaches are highly desirable. One direction in designing in-silico vaccine candidates may be based on exploiting the synonymous information, encoded in the viral genomes and related to gene expression, for attenuating the viral replication cycle while retaining its genotype and structure. The analysis and results reported here may thus have important implications in vaccine synthesis. Specifically, the outcomes of this study may provide clues and guidance into practical design of efficient and safe viral vaccines via attenuated viral material. Furthermore, it may also prove to be beneficial for other biotechnological objectives related to viral based products such as developing oncolytic viruses and engineering phages to fight bacteria. Indeed, we demonstrate, both in-vitro and in-vivo, how under-represented sequences can be utilized in order to obtain an attenuated Zika virus.

By a first aspect, there is provided a modified genome of an organism comprising at least one mutation, wherein the mutation generates a sequence that is underrepresented in the organism.

By another aspect, there is provided a method of generating a modified genome, the method comprising receiving a sequence of a genome of an organism, and introducing at least one mutation into the genome wherein the mutation generates a sequence that is underrepresented in the organism.

In some embodiments, the genome comprises at least one coding sequence. In some embodiments, the coding sequence encodes a protein. In some embodiments, the protein is selected from a functional category of proteins selected from surface proteins, structural proteins, enzymatic proteins, unclassified proteins and proteins of other functions. In some embodiments, the protein is not a surface protein. In some embodiments, the protein is not a structural protein. In some embodiments, the protein is an enzymatic protein. In some embodiments, the protein has an unknown or unclassified function. In some embodiments, the protein has a function other than surface, structural and enzymatic. In some embodiments, the protein is selected from a functional category of proteins selected from structural proteins, enzymatic proteins, unclassified proteins and proteins of other functions that are not surface proteins. In some embodiments, the mutation is within the coding sequence. In some embodiments, the coding sequence comprises the at least one mutation.

In some embodiments, at least one coding sequence comprises at least one mutation. In some embodiments, at least one coding sequence comprises a plurality of mutations. In some embodiments, at least one coding sequence comprises at least 1, 2, 3, 4, 5, 6, 7, 8, 9, 10, 20, 30, 40, 50, 60, 70, 80, 90, 95, 99, 100, 110, 120, 130, 140 150, 200, 250, 300, 350, 400, 450, 500, or 1000 mutations. Each possibility represents a separate embodiment of the invention. In some embodiments, a plurality of coding sequences comprises at least one mutation. In some embodiments, at least 1, 2, 3, 4, 5, 6, 7, 8, 9, 10, 20, 30, 40, 50, 60, 70, 80, 90, 95, 99, 100, 110, 120, 130, 140 150, 200, 250, 300, 350, 400, 450, 500, or 1000 coding sequences comprise at least one mutation. Each possibility represents a separate embodiment of the invention. In some embodiments, the coding sequence comprises at least 90 mutations. In some embodiments, the coding sequence comprises at least 100 mutations. In some embodiments, the coding sequence comprises at least 110 mutations. In some embodiments, the coding sequence comprises sufficient number of mutations to attenuate a pathogen comprising the coding sequence. In some embodiments, the pathogen is a virus.

In some embodiments, the coding sequence encodes a protein. In some embodiments, the coding sequence encodes a functional protein. In some embodiments, the coding sequence encodes a full protein. In some embodiments, the coding sequence encodes at least 10, 20, 30, 40, 50, 60, 70, 80, 90, 100, 150, 200, 250, 300, 350, 400, 450, 500, 550, 600, 650, 700, 750, 800, 850, 900, 950 or 1000 amino acids. Each possibility represents a separate embodiment of the invention. In some embodiments, the coding sequence encodes at least 500 amino acids. In some embodiments, the coding sequence encodes at least 900 amino acids.

In some embodiments, a sequence that is underrepresented in the organism is an underrepresented sequence. In some embodiments, the underrepresented sequence is underrepresented in the organism. In some embodiments, the underrepresented sequence is underrepresented in a genome of the organism. In some embodiments, the underrepresented sequence is underrepresented in an unmodified genome of the organism. In some embodiments, the underrepresented sequence is underrepresented in the genome of the organism before modification. In some embodiments, underrepresented is as compared to the representation that that would be present by chance. In some embodiments, underrepresented is as compared to an artificial random genomic sequence. In some embodiments, underrepresented is as compared to sequences in a host that can be infected by the organism. In some embodiments, the artificial random genomic sequence preserves amino acid sequence of the genome. In some embodiments, the artificial random genomic sequence preserves amino acid content of the genome. In some embodiments, the artificial random genomic sequence preserves codon usage in the genome. In some embodiments, the artificial random genomic sequence preserves codon usage bias in the genome. In some embodiments, the artificial random genomic sequence preserves dinucleotide distribution in the genome. In some embodiments, underrepresented sequences are determined by a method provided hereinbelow.

In some embodiments, the underrepresented sequence is a sequence of three nucleotides. In some embodiments, the underrepresented sequence is a sequence of four nucleotides. In some embodiments, the underrepresented sequence is a sequence of five nucleotides. In some embodiments, the underrepresented sequence is a sequence of three, four or five nucleotides. In some embodiments, the underrepresented sequence comprises 3-5 nucleotides. It will be understood that the sequence can be in any reading frame and thus can be anywhere within the genome or within a coding sequence. Indeed, a single mutation could generate several underrepresented sequences depending on the nucleotides around it. In some embodiments, the sequence is underrepresented in at least two reading frames. In some embodiments, the sequence is underrepresented in all three reading frames. In some embodiments, the sequence is a unique sequence. As used herein, a “unique sequence” refers to an underrepresented sequence that does not comprise another underrepresented sequence. Thus, for example AAA may be a unique underrepresented sequence, but if AAA is underrepresented in the organism than neither AAAA nor AAAAA are unique underrepresented sequence.

In some embodiments, the mutation is a point mutation. In some embodiments, the mutation changes one of the four DNA bases to a different base. In some embodiments, the mutation changes one of the four RNA bases to a different base. It will be understood that in a DNA genome the change will be to another DNA base and in an RNA genome the change will be to another RNA base. In some embodiments, the mutation is within a coding region and is a synonymous mutation. In some embodiments, a synonymous mutation mutates a codon to a synonymous codon. In some embodiments, a synonymous mutation does not alter an amino acid sequence encoded by the coding sequence comprising the mutation. In some embodiments, the mutated coding sequence encodes a protein with an identical amino acid sequence to the protein encoded by an unmutated coding sequence. In some embodiments, the mutated coding sequence comprises every synonymous mutation that increases the number of underrepresented sequences. In some embodiments, the mutated coding sequence comprises as many synonymous mutations as possible that increase the number of underrepresented sequences. In some embodiments, the mutated coding sequence comprises the number of synonymous mutations that produces the maximum number of underrepresented sequences. In some embodiments, the mutated coding sequence comprises the number of synonymous mutations sufficient to attenuate a pathogen comprising the coding sequence. In some embodiments, the pathogen is a virus. In some embodiments, a significant number is at least 90 synonymous mutations. In some embodiments, a significant number is at least 100 synonymous mutations. In some embodiments, a significant number is at least 110 synonymous mutations. In some embodiments, a significant number is at least 1% of all codons in the coding region. In some embodiments, a significant number is at least 5% of all codons in the coding region. In some embodiments, a significant number is at least 10% of all codons in the coding region. In some embodiments, a significant number is at least 1, 2, 3, 5, 7, 10, 15, 20, 25, 30, 35, 40, 45, or 50% of all codons in the coding region. Each possibility represents a separate embodiment of the invention.

In some embodiments, the mutation it is a silent mutation. In some embodiments, the mutation results in the alteration of an amino acid of the sequence encoded by the nuclei acid of the invention to an amino acid with a similar function characteristic. In some embodiments, a characteristic is selected from size, charge, isoelectric point, shape, hydrophobicity and structure. In some embodiments of the methods of the invention, the mutation results in a synonymous codon (Synonymous codons are provided in Table 7). In some embodiments, the mutation does not alter protein function. In some embodiments, the mutation alters protein function. As used herein, the term “silent mutation” refers to a mutation that does not affect or has little effect on protein functionality. A silent mutation can be a synonymous mutation and therefore not change the amino acids at all, or a silent mutation can change an amino acid to another amino acid with the same functionality or structure, thereby having no or a limited effect on protein functionality.

TABLE 7
synonymous codons

| | | | |
|---|---|---|---|
| F | UUC/UUU | P | CCC/CCU/CCA/CCG |
| L | CUC/UUG/CUU/CUG/ | T | ACC/ACU/ACA/ACG |
| | CUA/UUA | | |
| I | AUC/AUU/AUA | A | GCC/GCU/GCG/GCA |
| M | AUG | S | USS/UCU/UCA/UCG/ |
| | | | AGU/AGC |
| V | GUC/GUG/GUU/GUA | Q | CAA/CAG |
| Y | UAC/UAU | N | AAC/AAU |
| STOP | UAA/UAG/UGA | K | AAG/AAA |
| D | GAC/GAU | E | GAG/GAA |
| C | UGU/UGC | W | UGG |
| R | CGU/CGC/CGA/CGG/ | H | CAC/CAU |
| | AGG/AGA | | |
| G | GGU/GGC/GGG/GGA | | |

Introduction of a mutation into a genome is well known in the art. Any known genome editing method may be employed, so long as the mutation is specific to the location and change that is desired. Non-limiting examples of mutation methods include, site-directed mutagenesis, CRISPR/Cas9 and TALEN.

In some embodiments, the underrepresented sequence is a homooligonucleotide sequence. In some embodiments, the underrepresented sequence is not a homooligonucleotide sequence. As used herein, a “homooligonucleotide sequence” is a repeat sequence consisting of only a single type of bases, i.e., only A's, only T's, only C's or only G's. In some embodiments, the homooligonucleotide sequence consists of 3 nucleotides of the same nucleotide base. In some embodiments, the homooligonucleotide sequence consists of 4 nucleotides of the same nucleotide base. In some embodiments, the homooligonucleotide sequence consists of 5 nucleotides of the same nucleotide base. In some embodiments, the homooligonucleotide sequence consists of 3, 4 or 5 nucleotides of the same nucleotide base. In some embodiments, the homooligonucleotide sequence consists of 3 or 5 nucleotides of the same nucleotide base. It will be understood that a homooligonucleotide is by definition a palindromic sequence. In some embodiments, the underrepresented sequence is a palindromic sequence. In some embodiments, the underrepresented sequence is not a palindromic sequence. In some embodiments, the palindromic sequence is not an homooligonucleotide sequence. In some embodiments, the underrepresented sequence is selected from AAA, AAAA, AAAAA, TTT, TTTT, TTTTT, GGG, GGGG, GGGGG, CCC, CCCC, and CCCCC. It will be understood that in an RNA genome the Ts will be Us and so the sequences will be UUU, UUUU and UUUUU. In some embodiments, the underrepresented sequence is not any of AAA, AAAA, AAAAA, TTT, TTTT, TTTTT, GGG, GGGG, GGGGG, CCC, CCCC, and CCCCC. In some embodiments, the homooligonucleotide comprises only As. In some embodiments, the homooligonucleotide comprises only Ts. In some embodiments, the homooligonucleotide comprises only Gs. In some embodiments, the homooligonucleotide comprises only Cs.

In some embodiments, the underrepresented sequence is a palindromic sequence. As used herein, the term “palindromic sequence” refers to a sequence that is identical to its reverse compliment. In some embodiments, the underrepresented sequence is a sequence that is identical to its reverse complement. It will be understood by a skilled artisan that such a palindromic sequence can only have an even number of nucleotides as in a sequence with an odd number of nucleotides the middle nucleotide can never be identical to its reverse compliment. An example of a palindromic sequence would be GATC, as the reverse compliment is also GATC. Homooligonucleotides, therefore, cannot be palindromic sequences.

In some embodiments, the organism is a virus. In some embodiments, the virus is an RNA virus. In some embodiments, the virus is a DNA virus. In some embodiments, the DNA virus is a single-stranded virus. In some embodiments, the DNA virus is a double-stranded virus. In some embodiments, the double-stranded virus does not have an RNA stage. In some embodiments, the double-stranded virus does have an RNA stage. In some embodiments, the underrepresented sequence is underrepresented in the virus's genome. In some embodiments, the underrepresented sequence is underrepresented in the genomes of RNA viruses. In some embodiments, the underrepresented sequence is underrepresented in the genomes of DNA viruses. In some embodiments, the underrepresented sequence is underrepresented in the genomes of single-stranded viruses. In some embodiments, the underrepresented sequence is underrepresented in the genomes of double-stranded viruses. In some embodiments, the underrepresented sequence is underrepresented in a genome of a host organism. In some embodiments, the host is a host of the pathogen. In some embodiments, the host organism is the host of the virus. In some embodiments, the host organism is an organism infectable by the virus. In some embodiments, the underrepresented sequence is underrepresented in a genome of the virus and a genome of the host organism. In some embodiments, the underrepresented sequence is underrepresented in all three reading frames. In some embodiments, the virus is selected from the viruses provided in Supplementary Table S1 of Zarai et al., 2020, “Evolutionary selection against short nucleotide sequences in viruses and their related hosts”, DNA Res., Vol. 27: 2, herein incorporated by reference in its entirety. In some embodiments, the virus is ZIKA virus.

In some embodiments, WT ZIKA comprises the amino acid sequence provided in SEQ ID NO: 1. In some embodiments, WT ZIKA comprises a protein comprising the amino acid sequence of SEQ ID NO: 1. In some embodiments, WT ZIKA comprises a protein consisting of the amino acid sequence of SEQ ID NO: 1. In some embodiments, the protein is a polyprotein. In some embodiments, the WT ZIKA polyprotein comprises the amino acid sequence provided in SEQ ID NO: 1. In some embodiments, the WT ZIKA polyprotein consists of the amino acid sequence provided in SEQ ID NO: 1. In some embodiments, WT ZIKA is encoded by the nucleic acid sequence provided in SEQ ID NO: 2. In some embodiments, WT ZIKA is encoded by a nucleic acid sequence comprising the sequence provided in SEQ ID NO: 2. In some embodiments, the WT ZIKA genome comprises SEQ ID NO: 2. In some embodiments, WT ZIKA polyprotein is encoded by the nucleic acid sequence provided in SEQ ID NO: 2. In some embodiments, SEQ ID NO: 1 is encoded by the nucleic acid sequence provided in SEQ ID NO: 2.

In some embodiments, a mutant ZIKA virus of the invention comprises the amino acid sequence provided in SEQ ID NO: 1. In some embodiments, a mutant ZIKA virus of the invention comprises a protein comprising the amino acid sequence provided in SEQ ID NO: 1. In some embodiments, a mutant ZIKA virus of the invention comprises a protein consisting of the amino acid sequence provided in SEQ ID NO: 1. In some embodiments, a mutant ZIKA polyprotein comprises the amino acid sequence of SEQ ID NO: 1. In some embodiments, a mutant ZIKA polyprotein consists of the amino acid sequence of SEQ ID NO: 1. In some embodiments, a mutant ZIKA is encoded by the nucleic acid sequence of SEQ ID NO: 3. In some embodiments, a WT ZIKA polyprotein is encoded by the nucleic acid sequence of SEQ ID NO: 3. In some embodiments, SEQ ID NO: 1 is encoded by SEQ ID NO: 3. Since SEQ ID NO: 3 comprises only synonymous mutations it encodes the same amino acid sequence, SEQ ID NO: 1, as the WT genomic sequence (SEQ ID NO: 2). In some embodiments, a mutant nucleotide sequence encoding an attenuated ZIKA or a polyprotein of an attenuated ZIKA is generated by mutating codons 3, 4, 7, 8, 15, 16, 22, 43, 44, 47, 57, 65, 68, 84, 116, 125, 126, 142, 143, 161, 163, 164, 168, 172, 195, 197, 198, 205, 208, 211, 222, 262, 263, 267, 303, 320, 325, 336, 338, 339, 354, 355, 370, 378, 382, 393, 394, 410, 413, 434, 435, 445, 480, 485, 489, 507, 508, 511, 512, 513, 515, 520, 548, 552, 553, 564, 565, 567, 579, 593, 606, 607, 610, 617, 618, 631, 638, 649, 668, 669, 679, 680, 741, 742, 775, 776, 790, 794, 810, 812, 813, 817, 836, 837, 851, 858, 859, 872, and 901, of SEQ ID NO: 2. In some embodiments, SEQ ID NO: 3 is generated by mutating codons 3, 4, 7, 8, 15, 16, 22, 43, 44, 47, 57, 65, 68, 84, 116, 125, 126, 142, 143, 161, 163, 164, 168, 172, 195, 197, 198, 205, 208, 211, 222, 262, 263, 267, 303, 320, 325, 336, 338, 339, 354, 355, 370, 378, 382, 393, 394, 410, 413, 434, 435, 445, 480, 485, 489, 507, 508, 511, 512, 513, 515, 520, 548, 552, 553, 564, 565, 567, 579, 593, 606, 607, 610, 617, 618, 631, 638, 649, 668, 669, 679, 680, 741, 742, 775, 776, 790, 794, 810, 812, 813, 817, 836, 837, 851, 858, 859, 872, and 901, of SEQ ID NO: 2. In some embodiments, SEQ ID NO: 3 provides a nucleic acid molecule of an exemplary modified genome produced by a method of the invention.

In some embodiments, the organism is a host of a virus. In some embodiments, the organism is a host of a double-stranded DNA virus. In some embodiments, the host is a bacterium. In some embodiments, the host is a bacterium infected by a double stranded DNA virus. In some embodiments, the host is a vertebrate. In some embodiments, the host is a mammal. In some embodiments, the mammal is a human. In some embodiments, the host is selected from the hosts provided in Supplementary Table 51 of Zarai et al., 2020, “Evolutionary selection against short nucleotide sequences in viruses and their related hosts”, DNA Res., Vol. 27: 2, herein incorporated by reference in its entirety.

In some embodiments, the host is a bacterium and the underrepresented sequence is a palindromic sequence. In some embodiments, the host is a bacterium that can be infected by a double-stranded DNA virus and the underrepresented sequence is a palindromic sequence. In some embodiments, the organism is a virus and the underrepresented sequence is a palindromic sequence. In some embodiments, the organism is a DNA virus and the underrepresented sequence is a palindromic sequence. In some embodiments, the organism is a double-stranded DNA virus and the underrepresented sequence is a palindromic sequence. In some embodiments, the organism is a double-stranded DNA virus that infects bacteria and the underrepresented sequence is a palindromic sequence. In some embodiments, the organism is a bacteriophage and the underrepresented sequence is a palindromic sequence.

In some embodiments, the underrepresented sequence is selected from Table 1. In some embodiments, the underrepresented sequence is selected from TTT, AAA, TAG, CCC, GAC, GGG, AAAA, TTTT, GATC, CGCG, GGGG, CCCC, AAAAA, TTTTT, GGATC, GATCT, GGGGG, and CCCCC. In some embodiments, the underrepresented sequence is selected from TTT, AAA, TAG, CCC, GAC, GGG, AAAA, TTTT, CGCG, GGGG, CCCC, AAAAA, TTTTT, GGATC, GATCT, GGGGG, and CCCCC. In some embodiments, the underrepresented sequence is selected from TTT, AAA, TAG, CCC, GAC, GGG, AAAA, TTTT, GGGG, CCCC, AAAAA, TTTTT, GGATC, GATCT, GGGGG, and CCCCC. In some embodiments, the underrepresented sequence is selected from TAG, GAC, GATC, CGCG, GGATC, and GATCT. In some embodiments, the underrepresented sequence is selected from TAG, GAC, GGATC, and GATCT. In some embodiments, all the underrepresented sequences incorporated into the modified genome are selected from the groups provided herein. In some embodiments, at least one of the underrepresented sequences incorporated into the modified genome is selected from the groups provided herein. In some embodiments, the modified genome comprises mutation to include at least one of TAG, GAC, GGATC, and GATCT.

In some embodiments, the underrepresented sequence is selected from Table 2. In some embodiments, the underrepresented sequence is not selected from Table 2. In some embodiments, the underrepresented sequence is selected from AAAA, AAAAA, TTTT, TTTTT, CCCC, CCCCC, CGGA, ACCAA, and GATC. In some embodiments, the underrepresented sequence is selected from AAAAA, CCCC, GCGA, CAATC, GGGG, TTGGA. In some embodiments, the organism is a Pl-ssRNA virus and the underrepresented sequence is AAAA or AAAAA. In some embodiments, the organism is a Pl-ssDNA virus and the underrepresented sequence is TTTT. In some embodiments, the organism is a Me-ssRNA virus and the underrepresented sequence is AAAA or TTTTT. In some embodiments, the organism is a Me-ssRNA virus and the underrepresented sequence is AAAA, AAAAA or TTTTT. In some embodiments, the organism is a Ve-ssRNA virus and the underrepresented sequence is AAAAA or TTTT. In some embodiments, the organism is a Ve-ssRNA virus and the underrepresented sequence is AAAAA, CCCC or TTTT. In some embodiments, the organism is a Ve-dsDNA virus and the underrepresented sequence is CCCC or CCCCC. In some embodiments, the organism is a Ve-dsDNA virus and the underrepresented sequence is CCCC, GCGA, CAATC or CCCCC. In some embodiments, the organism is a Ve-ssDNA virus and the underrepresented sequence is CGGA or ACCAA. In some embodiments, the organism is a Ba-dsDNA virus and the underrepresented sequence is GATC or AAAAA. In some embodiments, the organism is a Ba-dsDNA virus and the underrepresented sequence is GATC, GGGG, TTGGA or AAAAA. In some embodiments, the organism is a Fu-dsDNA virus and the underrepresented sequence is AAAA. In some embodiments, the underrepresented sequence is selected from GCGA, CAATC, ACCAA and TTGGA. In some embodiments, the modified genome comprises mutation to include at least one of GCGA, CAATC, ACCAA and TTGGA.

In some embodiments, the underrepresented sequence is not selected from the list provided in Table 4. Table 4 provides examples of underrepresented sequences produced by random models. Notably the sequences are not common between reading frames and thus they are not genuine underrepresented sequences. In some embodiments, the underrepresented sequence is a common underrepresented sequence. n some embodiments, the underrepresented sequence is underrepresented in all three reading frames. In some embodiments, a sequence underrepresented in only one reading frame is not a genuine underrepresented sequence. In some embodiments, a sequence underrepresented in only two reading frames is not a genuine underrepresented sequence. In some embodiments, the underrepresented sequence is not any of AGGGG, GATAT, AAACA, CTCGA, GGGCT, AGCCT, TCTTT, GGGTG, CACTT, GGACT, GGCTC, TGGTT, CGAGA, GATCG, TATGT, GTGGC, CAAAA, CAGGG and TATTG. In some embodiments, the underrepresented sequence is not selected from the list provided in Table 6. In some embodiments, the underrepresented sequence is not any of GCA, CGA, GGC, GGT, ATA, GGG, CTG, TCA, CGA, TGC, GTC, CAG, TGT, TGC, TTC, TAG, TAT, TGA, CGT, GCA, TCA, CGA, TTA, AAC, TCG, TGC, AGT, AAA, GCC, TTG, CCT, GCA, GTA, AGC, GGC, ATC, CTC, AAG, GGG, GAT, AGT, GGT, CTT, ATA, GTA, GCC, ATC, GTC, TCG, GGG, TTG, TAA, TCA, CGA, TAC, TGC, CAG, CGG, GGG, TGT, ATT, GGG, GCA, GTA, GGC, GAG, ACT, GGT, CTT, GTA, GCC, ATC, AGG, GGG, CTG, GTG, TTG, TAA, TCA, CGA, TAC, TGC, CAG, GGG, TGT, GCA, CGA, GTA, ACC, ATC, CAG, CCG, ACT, AGT, GGT, GCA, GTA, GCC, ATC, GTC, GGG, CTG, TTG, GTT TAA, TCA, CGA, TGC, CAG, TTG, GAT, ACA, CTA, TAC, TGC, GAG, AGG, GAT, TCT, GGT, GTT, CAA, TGA, ATA, TAC, GTC, TCG, TTG, TAT, ACT, AGT, CCA, ATG, TAT, CAA, AGC, GAG, GCT, AGT, GGT, CTT, GTT, GTA, GGC, GTC, ACG, CTG, TTG, AAT, TAT, CAA, CGA, TGA, ATA, TTA, CAC, AGC, GGT. In some embodiments, the virus is a virus from Table 6 and the underrepresented sequence is not one provided for that virus in Table 6.

In some embodiments, the underrepresented sequence is selected from the list provided in Supplementary Table S3 of Zarai et al., 2020. In some embodiments, the underrepresented sequence is selected from the list provided in Supplementary Table S4 of Zarai et al., 2020. In some embodiments, the underrepresented sequence is selected from the list provided in Supplementary Table S5 of Zarai et al., 2020. In some embodiments, the underrepresented sequence is selected from the list provided in Supplementary Table S6 of Zarai et al., 2020. In some embodiments, the underrepresented sequence is selected from the list provided in Supplementary Table S7 of Zarai et al., 2020.

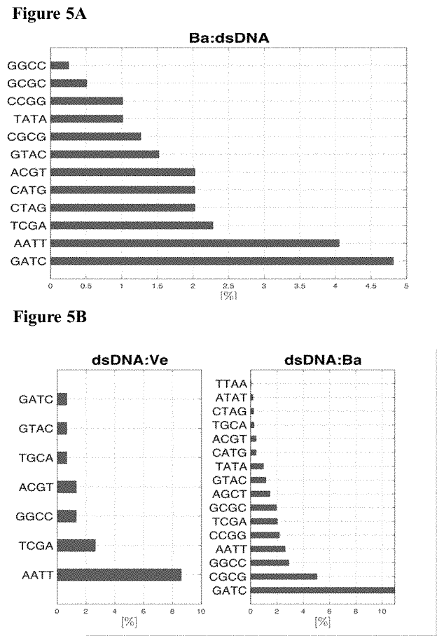

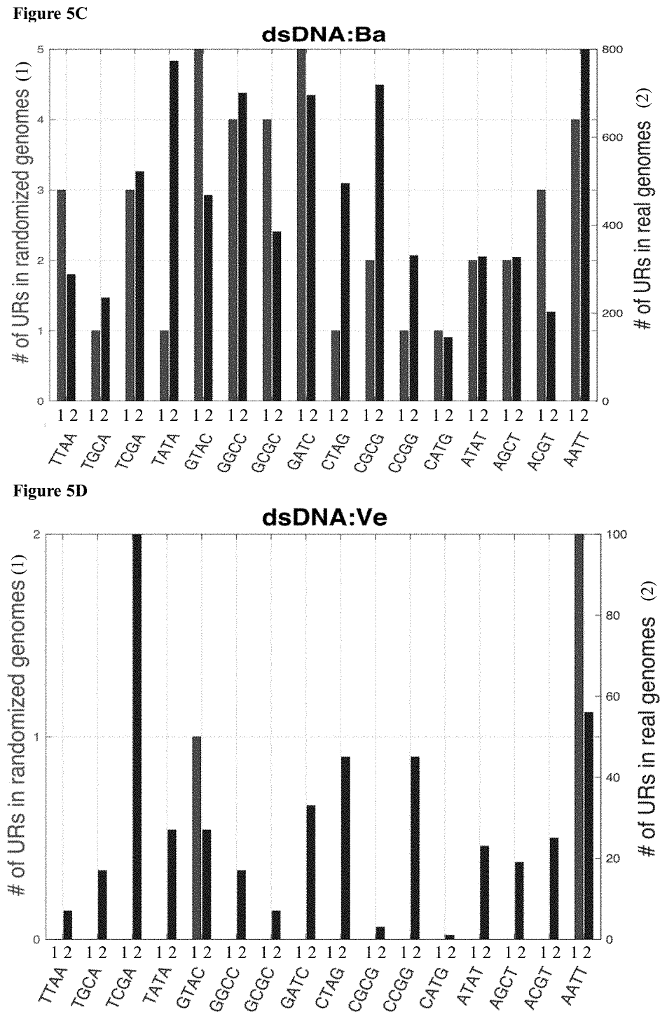

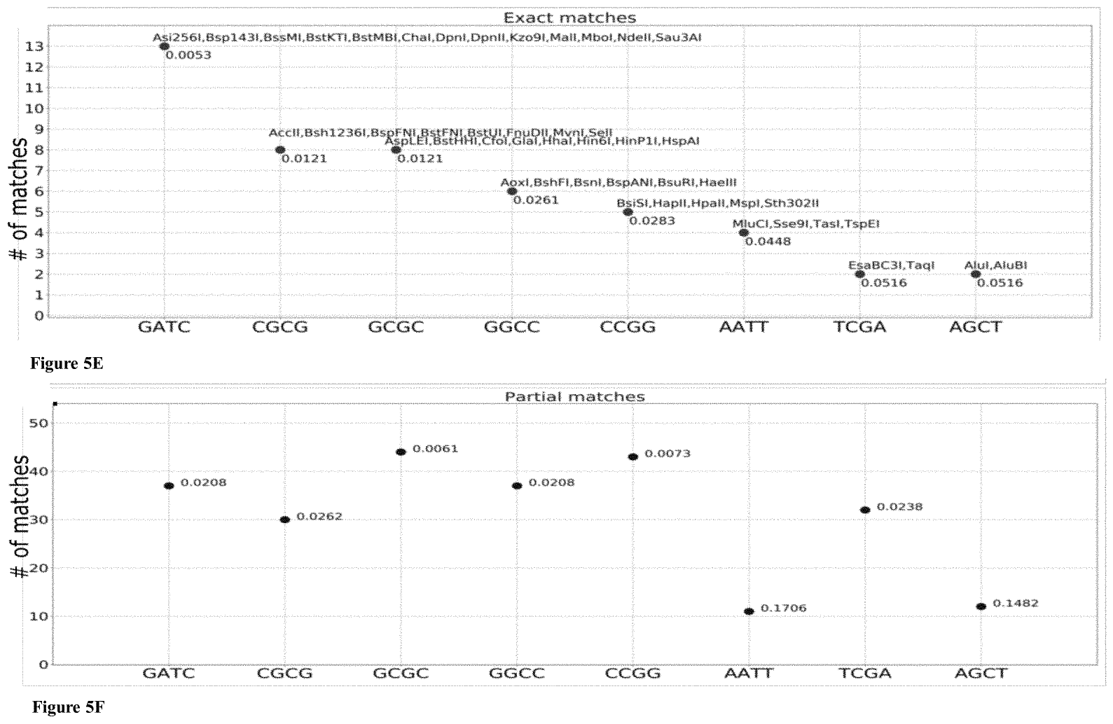

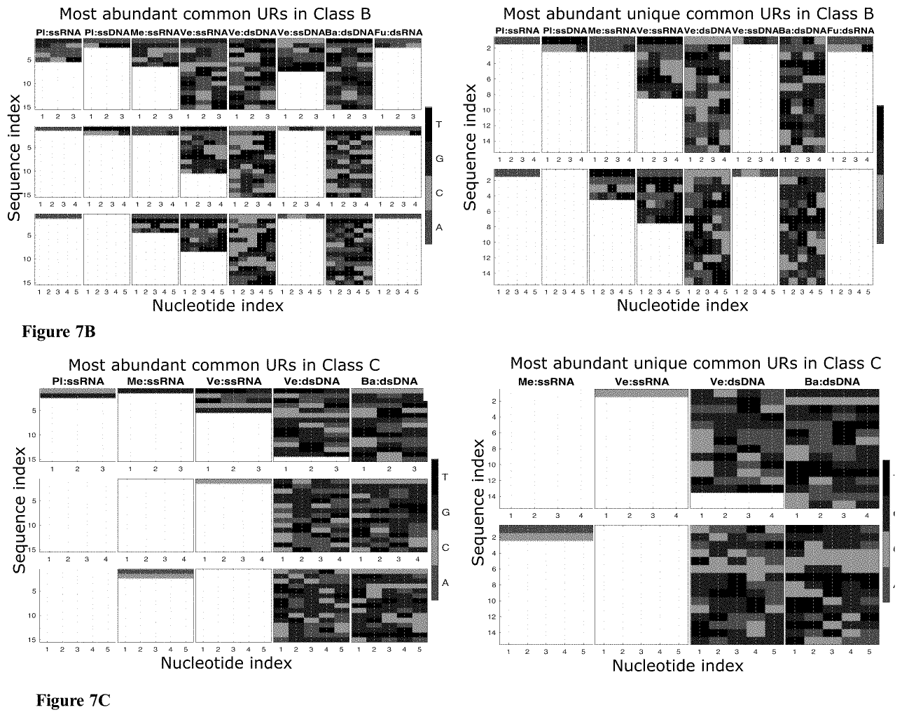

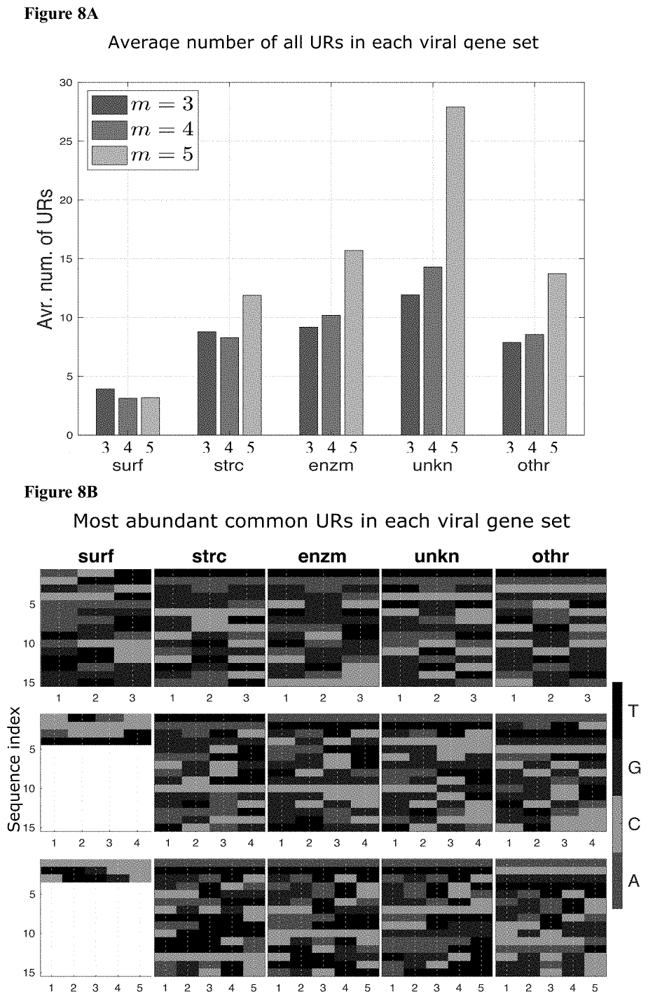

In some embodiments, the underrepresented sequence is provided in FIG. 5A. In some embodiments, the underrepresented sequence is provided in FIG. 5B. In some embodiments, the underrepresented sequence is provided in FIG. 5C. In some embodiments, the underrepresented sequence is provided in FIG. 5D. In some embodiments, the underrepresented sequence is provided in FIG. 7B. In some embodiments, the underrepresented sequence is provided in FIG. 7C. In some embodiments, the underrepresented sequence is provided in FIG. 8B.

In some embodiments, underrepresented palindromic sequences in bacteria that are hosts to double stranded DNA viruses are provided in FIG. 5A. In some embodiments, underrepresented palindromic sequences in bacteria that are hosts to double stranded DNA viruses are selected from GGCC, GCGC, CCGG, TATA, CGCG, GTAC, ACGT, CATG, CTAG, TCGA, AATT and GATC. In some embodiments, underrepresented palindromic sequences in double stranded DNA viruses that infect bacteria (that is that are bacteriophages) are provided in FIG. 5B on the right. In some embodiments, underrepresented palindromic sequences in double stranded DNA viruses that infect bacteria (that is that are bacteriophages) are selected from TTAA, ATAT, CTAG, TGCA, ACGT, CATG, TATA, GTAC, AGCT, GCGC, TCGA, CCGG, AATT, GGCC, CGCG, and GATC. In some embodiments, underrepresented palindromic sequences in double stranded DNA viruses that infect bacteria (that is that are bacteriophages) are provided in FIG. 5C on the right. In some embodiments, underrepresented palindromic sequences in double stranded DNA viruses that infect vertebrates are provided in FIG. 5B on the left. In some embodiments, underrepresented palindromic sequences in double stranded DNA viruses that infect vertebrates are selected from GATC, GTAC, TGCA, ACGT, GGCC, TCGA, and AATT. In some embodiments, underrepresented palindromic sequences in double stranded DNA viruses that infect vertebrates are provided in FIG. 5D on the left. In some embodiments, underrepresented palindromic sequences in double stranded DNA viruses that infect vertebrates are selected from TTAA, TGCA, TCGA, TATA, GTAC, GGCC, GCGC, GATC, CTAG, CGCG, CCGG, CATG, ATAT, AGCT, ACGT and AATT. In some embodiments, the palindromic sequence is not GATC.

In some embodiments, the coding sequence encodes a protein with a function, and the underrepresented sequence is underrepresented in genes encoding proteins with that function. In some embodiments, the function is a surface protein. In some embodiments, the function is not a surface protein. In some embodiments, the function is a structural protein. In some embodiments, the function is an enzymatic protein. In some embodiments, the function is an unclassified and/or unknown function. In some embodiments, the function is any other function. In some embodiments, any other function is a known function other than surface, structural, and enzymatic.

In some embodiments, the protein is a surface protein and the underrepresented sequence is selected from ACT, CTT, GAC, CCT, AAA, AGG, AAT, AGT, TGA, ACC, GTC, TTC, TAG, GGG, GTG, CTAC, CCCC, ACCT, TTTT, CCCCC, TTGCC, and CTTGC. In some embodiments, the protein is a surface protein and the underrepresented sequence is selected from ACT, CTT, GAC, CCT, AGG, AAT, AGT, TGA, ACC, GTC, TTC, TAG, GTG, CTAC, ACCT, CCCCC, TTGCC, and CTTGC.

In some embodiments, the protein is a structural protein and the underrepresented sequence is selected from TTT, AAA, GAG, GGG, GGA, CCC, GCT, GCC, CGA, GTC, AGG, CTC, TGT, GAC, AAT, TTTT, AAAA, GATC, GGGG, GGCT, AATT, CGCG, AGCT, GCTT, CCCC, GGAG, GTAC, AAAT, AGCC, TCAG, AAAAA, TTTTT, GGATC, GATCA, CCTGG, AAATT, CGCGC, TTTTC, AATTT, CTTCA, CCCCC, AGATC, and AGCTC. In some embodiments, the protein is a structural protein and the underrepresented sequence is selected from GAG, GGA, GCT, GCC, CGA, GTC, AGG, CTC, TGT, GAC, AAT, GATC, GGCT, AATT, CGCG, AGCT, GCTT, GGAG, GTAC, AAAT, AGCC, TCAG, GGATC, GATCA, CCTGG, AAATT, CGCGC, TTTTC, AATTT, CTTCA, AGATC, and AGCTC. In some embodiments, the protein is a structural protein and the underrepresented sequence is selected from TTT, AAA, GAG, GGG, GGA, CCC, GCT, GCC, CGA, GTC, AGG, CTC, TGT, GAC, AAT, TTTT, AAAA, GGGG, GGCT, GCTT, CCCC, GGAG, AAAT, AGCC, TCAG, AAAAA, TTTTT, GGATC, GATCA, CCTGG, AAATT, CGCGC, TTTTC, AATTT, CTTCA, CCCCC, AGATC, and AGCTC. In some embodiments, the protein is a structural protein and the underrepresented sequence is selected from GAG, GGA, GCT, GCC, CGA, GTC, AGG, CTC, TGT, GAC, AAT, GGCT, GCTT, GGAG, AAAT, AGCC, TCAG, GGATC, GATCA, CCTGG, AAATT, CGCGC, TTTTC, AATTT, CTTCA, AGATC, and AGCTC. In some embodiments, a structural protein is a not a surface protein. In some embodiments, a structural protein is a not an enzymatic protein.

In some embodiments, the protein is an enzymatic protein and the underrepresented sequence is selected from TTT, AAA, GAG, GGA, CGC, TGT, GGG, AAT, GCT, TAG, AGG, CGA, GTC, GAC, CCC, AAAA, TTTT, GATC, CGCG, AATT, AGCT, GGCT, TCGA, GGGG, CCCC, GGCC, GCGC, GGAG, GTAC, TTGG, AAAAA, TTTTT, GATCT, AGATC, GGATC, GATCA, CCTGG, AAATT, AATTT, CCCCC, CGATC, TTCGA, CTTGG, TTTTC, and AGCTT. In some embodiments, the protein is an enzymatic protein and the underrepresented sequence is selected from GAG, GGA, CGC, TGT, AAT, GCT, TAG, AGG, CGA, GTC, GAC, GATC, CGCG, AATT, AGCT, GGCT, TCGA, GGCC, GCGC, GGAG, GTAC, TTGG, GATCT, AGATC, GGATC, GATCA, CCTGG, AAATT, AATTT, CGATC, TTCGA, CTTGG, TTTTC, and AGCTT. In some embodiments, the protein is an enzymatic protein and the underrepresented sequence is selected from TTT, AAA, GAG, GGA, CGC, TGT, GGG, AAT, GCT, TAG, AGG, CGA, GTC, GAC, CCC, AAAA, TTTT, GGCT, GGGG, CCCC, GGAG, TTGG, AAAAA, TTTTT, GATCT, AGATC, GGATC, GATCA, CCTGG, AAATT, AATTT, CCCCC, CGATC, TTCGA, CTTGG, TTTTC, and AGCTT. In some embodiments, the protein is an enzymatic protein and the underrepresented sequence is selected from GAG, GGA, CGC, TGT, AAT, GCT, TAG, AGG, CGA, GTC, GAC, GGCT, GGAG, TTGG, GATCT, AGATC, GGATC, GATCA, CCTGG, AAATT, AATTT, CGATC, TTCGA, CTTGG, TTTTC, and AGCTT.

In some embodiments, the protein has an unknown or uncharacterized function and the underrepresented sequence is selected from TTT, AAA, TAG, CCC, TGT, CGC, GAC, GGG, AAT, ACA, CTC, GAG, GTC, GCT, GGA, AAAA, TTTT, GATC, CCCC, GGCC, CGCG, GGGG, AATT, AGCT, CCGG, GGCT, GCGC, GTAC, AGCC, TCGA, AAAAA, TTTTT, AGATC, GATTCT, GATCA, GGATC, CCCCC, AATTT, CCTGG, GGGGG, AAAAT, AAATT, TGGCT, AGCTT, and ATTTT. In some embodiments, the protein has an unknown or uncharacterized function and the underrepresented sequence is selected from TAG, TGT, CGC, GAC, AAT, ACA, CTC, GAG, GTC, GCT, GGA, GATC, GGCC, CGCG, AATT, AGCT, CCGG, GGCT, GCGC, GTAC, AGCC, TCGA, AGATC, GATTCT, GATCA, GGATC, AATTT, CCTGG, AAAAT, AAATT, TGGCT, AGCTT, and ATTTT. In some embodiments, the protein has an unknown or uncharacterized function and the underrepresented sequence is selected from TTT, AAA, TAG, CCC, TGT, CGC, GAC, GGG, AAT, ACA, CTC, GAG, GTC, GCT, GGA, AAAA, TTTT, CCCC, GGGG, GGCT, AGCC, AAAAA, TTTTT, AGATC, GATTCT, GATCA, GGATC, CCCCC, AATTT, CCTGG, GGGGG, AAAAT, AAATT, TGGCT, AGCTT, and ATTTT. In some embodiments, the protein has an unknown or uncharacterized function and the underrepresented sequence is selected from TAG, TGT, CGC, GAC, AAT, ACA, CTC, GAG, GTC, GCT, GGAGGCT, AGCC, AGATC, GATTCT, GATCA, GGATC, AATTT, CCTGG, AAAAT, AAATT, TGGCT, AGCTT, and ATTTT.

In some embodiments, the protein has another function and the underrepresented sequence is selected from TTT, AAA, CCC, GGG, ACT, GAC, TAG, CTC, GGA, CGC, GCG, GTG, GAG, AGT, CGA, AAAA, GATC, TTTT, GGGG, CCCC, AATT, CGCG, GCGC, GGAG, AGCT, GGCT, TCGA, GTAC, GGCC, CTCC, AAAAA, CCCCC, GGGGG, TTTTT, GATCT, AGATC, GGATC, GATCA, CCTGG, CGCGG, CCCCA, AAAAT, CGCGC, GGAGC, and GGCGC. In some embodiments, the protein has another function and the underrepresented sequence is selected from ACT, GAC, TAG, CTC, GGA, CGC, GCG, GTG, GAG, AGT, CGA, GATC, AATT, CGCG, GCGC, GGAG, AGCT, GGCT, TCGA, GTAC, GGCC, CTCC, GATCT, AGATC, GGATC, GATCA, CCTGG, CGCGG, CCCCA, AAAAT, CGCGC, GGAGC, and GGCGC. In some embodiments, the protein has another function and the underrepresented sequence is selected from TTT, AAA, CCC, GGG, ACT, GAC, TAG, CTC, GGA, CGC, GCG, GTG, GAG, AGT, CGA, AAAA, TTTT, GGGG, CCCC, GGAG, GGCT, GGCC, CTCC, AAAAA, CCCCC, GGGGG, TTTTT, GATCT, AGATC, GGATC, GATCA, CCTGG, CGCGG, CCCCA, AAAAT, CGCGC, GGAGC, and GGCGC. In some embodiments, the protein has another function and the underrepresented sequence is selected from ACT, GAC, TAG, CTC, GGA, CGC, GCG, GTG, GAG, AGT, CGA, GGAG, GGCT, GGCC, CTCC, GATCT, AGATC, GGATC, GATCA, CCTGG, CGCGG, CCCCA, AAAAT, CGCGC, GGAGC, and GGCGC. In some embodiments, another function which is a known and/or characterized function and is not a surface protein, or an enzymatic protein.

In some embodiments, the method comprises selecting at least one coding sequence within the genome. In some embodiments, the method comprises introducing at least one mutation into the at least one coding sequence. In some embodiments, the method comprises generating at least one mutation in the at least one coding sequence.

In some embodiments, the method is an in vitro method. In some embodiments, the method is an ex vivo method. In some embodiments, the modified genome is on a plasmid. In some embodiments, the modified genome is outside of an organism.

According to another aspect, there is provided a modified genome produced by a method of the invention.

According to another aspect, there is provided an organism comprising a modified genome of the invention.

According to another aspect, there is provide a cell comprising a modified genome of the invention.

In some embodiments, the organism is a virus. In some embodiments, the virus is an attenuated virus. In some embodiments, the virus is an attenuated live virus. In some embodiments, the virus is an attenuated dead virus. In some embodiments, the attenuated virus is a vaccine. In some embodiments, the attenuated virus is less virulent than the unattenuated virus. In some embodiments, the attenuated virus replicates more slowly than the unattenuated virus. As used herein, the term “attenuated virus” refers to a virus, in which the virulence thereof has been reduced, e.g., by genetic manipulation of the viral genome.

In some embodiments, the organism is a bacterium. In some embodiments, the cell is a bacterial cell. In some embodiments, the cell is a prokaryotic cell. In some embodiments, the cell is a eukaryotic cell. In some embodiments, the cell is a mammalian cell. In some embodiments, the cell is a human cell.

According to another aspect, there is provide a pharmaceutical composition comprising a modified genome of the invention.

According to another aspect, there is provide a pharmaceutical composition comprising a cell of the invention.

According to another aspect, there is provide a pharmaceutical composition comprising an organism of the invention.

In some embodiments, the pharmaceutical composition further comprises a pharmaceutically acceptable carrier, excipient or adjuvant. In some embodiments, the pharmaceutical composition is a vaccine composition. In some embodiments, the pharmaceutical composition is configured for administration to a subject. In some embodiments, the pharmaceutical composition is configured for systemic administration.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal.

The dosage administered will be dependent upon the age, health, and weight of the recipient, kind of concurrent treatment, if any, frequency of treatment, and the nature of the effect desired.

According to another aspect, there is provide a computer program product comprising a non-transitory computer-readable storage medium having program code embodied thereon, the program code executable by at least one hardware processor to:
a. receive a sequence of a genome of an organism;
b. receive a list of underrepresented sequences of length 3, 4 or 5 nucleotides in the organism;
c. calculate mutations within the genome that generate at least one underrepresented sequence from the list; and
d. provide an output modified genome comprising at least one calculated mutation.

In some embodiments, the computer program product is for generating a modified genome. In some embodiments, the modified genome is a modified genome of the invention. In some embodiments, the computer program product calculates mutation within at least one coding sequence of the genome. In some embodiments, the computer program product provides an output modified genome comprising the at least one coding sequence comprising at least one calculated mutation.

In some embodiments, the at least one calculated mutation does not alter an amino acid sequence of a protein encoded by the at least one coding sequence. In some embodiments, the computer program product calculates synonymous mutation within the at least one coding region. In some embodiments, the output modified genome comprises at least one calculated synonymous mutation.

In some embodiments, the output modified genome comprises the at least one coding sequence comprising every possible calculated synonymous mutation. In some embodiments, the list of underrepresented sequences is ranked in order of the extent of underrepresentation in the genome. In some embodiments, the output modified genome comprises the 1, 2, 3, 4, 5, 6, 7, 8, 9, 10, 15, 20, 25, 30, 35, 40, 45, 50, 60, 70, 80, 90 or 100 most highly underrepresented sequences that could be generated by a synonymous mutation. Each possibility represents a separate embodiment of the invention. In some embodiments, the calculated mutations are ranked by the extent of underrepresentation of the sequence produced by the mutation. In some embodiments, the output modified genome comprises the 1, 2, 3, 4, 5, 6, 7, 8, 9, 10, 15, 20, 25, 30, 35, 40, 45, 50, 60, 70, 80, 90 or 100 most highly ranked mutations. Each possibility represents a separate embodiment of the invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement one or more of the disclosed embodiments described herein. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

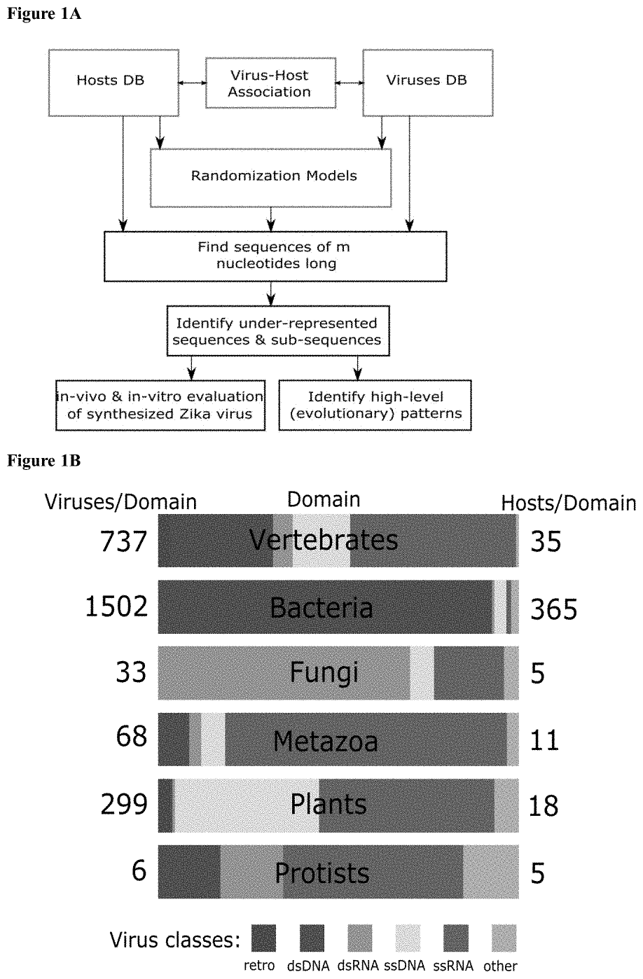

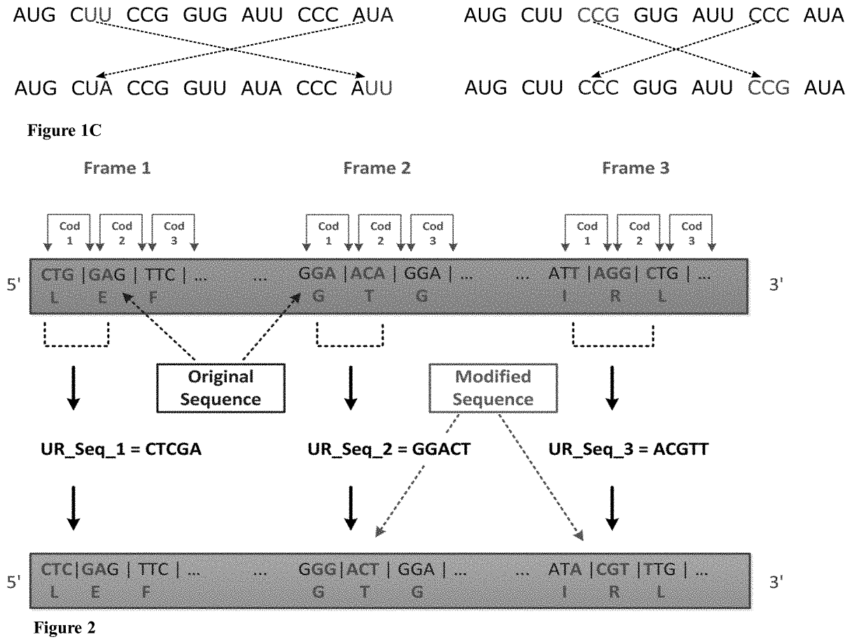

Analysis Flow Overview: The general flow of our analysis is depicted in FIG. 1A. The dataset of virus-host associations was retrieved from Mihara et al, “Linking virus genomes with host taxonomy”, Viruses, 8, 66, herein incorporated by reference in its entirety. This included 2,625 unique viruses and 439 corresponding hosts, where all the corresponding coding sequences were downloaded and processed. Randomization models were used to generate many random variants of the host and virus coding sequences. Two different randomization models were employed, each control for different biases. A dinucleotide randomization model preserves both amino-acid order and content and the distribution of all 16 possible pairs of nucleotides, whereas a synonymous codon randomization model preserves both amino-acid order and content, and the codon usage bias. These were then used to statistically infer short nucleotide sequences that are under-represented within both the original host and virus genome coding regions, in each reading frame, and those that are common to all three reading frames. These under-represented sequences were analyzed and compared among different viral groups and viral proteins.

Database: The virus and host coding sequences and association information was retrieved from Goz et al., Universal evolutionary selection for high dimensional silent patterns of information hidden in the redundancy of viral genetic code, Bioinformatics, herein incorporated by reference in its entirety. In brief, the association between viruses and hosts was derived from the GenomeNet Virus-Host Database. The database contains 2,625 unique viruses with a total of 147,286 coding sequences and 439 corresponding unique hosts from all kingdoms of life. FIG. 1B depicts the six host domains in the database (vertebrates, bacteria, fungi, metazoa, planta and protists), where we specify for each host domain the portion of the corresponding viruses belonging to each virus type. The virus types in the database are reverse-transcribing (retro), double-stranded DNA (dsDNA), double-stranded RNA (dsRNA), single-stranded DNA (ssDNA), single stranded RNA (ssRNA, positive and negtabative sense) and other (unclassified).

Randomization Models and Statistical Analysis. The question that we must first address is: what constitutes an under-represented sequence in a coding region? In order to detect sequences that are statistically under-represented in the coding regions, our statistical background model must capture well-understood coding region features, which are known to be under selection. For example, selection for codon usage bias may cause few short sequences to be in low abundance in the coding regions (as opposed, for example, to regions that are not translated). This, however, doesn't imply that these short sequences were directly selected against by evolutionary forces. Our definition of under-represented short nucleotide sequences in the coding region must then be formulated with respect to all known coding region features (i.e., amino-acids content and order, codon usage bias and dinucleotide distribution), in order to suggest possibly new evolutionary forces acting on the viral coding regions.

To that end, two randomization models were used to evaluate our hypothesis for short, under-represented nucleotide sequences in the coding regions of the viruses and in the coding regions of their corresponding hosts. The first, called dinucleotide randomization, preserves both amino acid order and content (and thus the resulting protein), and the frequencies of the 16 possible pairs of adjacent nucleotides (dinucleotides). The second, called synonymous codon randomization preserves both amino-acids order and content (and thus the resulting protein) and the codon usage bias. FIG. 1C depicts a schematic description of both randomization methods.

A selection against short nucleotide sequences that cannot be explained by the canonical genomic features that are preserved by both randomization models implies that these sequences will appear more frequently in the random variants (generated by the above randomization models) than in the original genome. Empirical p-values were derived from the empirical null model defined by the above two randomization models. The p-value estimates the probability of obtaining a random value (i.e., the number of occurrences of a sequence in the coding regions) that is the same or larger than the observed value in the original genome. This was performed separately in each of the three reading frames. A sequence was declared under-represented, if its p-values corresponding to both randomization models were both less than or equal to 0.05. Note that in the case of synonymous codon randomization, no under-represented sequence of size three nucleotides can be identified in the first reading frame.

Specifically, when analyzing under-represented sequences in the viruses, we compared the original genome to 1,000 corresponding randomization variants generated by each of the randomization models described above. Under-represented sequences were then identified separately in each reading frame. In addition, common under-represented nucleotide sequences were identified (i.e., sequences that are under-represented in all three reading frames—see Materials and Methods). This may indicate selection against sequences that may “interfere” with the process of mRNA translation. See Materials and Methods for an additional method of identifying under-represented sequences in the viruses based on the corresponding hosts (i.e., host-based as opposed to random-based analysis).

Due to the large size of the host genome, the analysis of under-represented sequences in the hosts was performed differently than in the viruses. Instead, the hosts were analyzed relative to their corresponding viruses. Recall that a host can be infected by several viruses. Specifically, for each pair of a host and a corresponding virus (i.e., a virus that infects the host), we randomly sampled the host coding sequences with a sample size equals the total size of the virus coding sequences. Twenty samples were used for each host-virus pair. Each sample was compared to 1,000 corresponding randomization variants generated by each of the random models. Thus, twenty sets of under-represented sequences were identified in the host, for each reading frame, given a corresponding virus. A sequence that is under-represented in at least ten of the twenty samples, per reading frame, is then considered as under-represented in the host, given the corresponding virus. This is referred to as the “sampled majority under-represented set” of the host given a corresponding virus (see Materials and Methods below). The final set of sequences that are under-represented in the host was defined by the intersection over all the corresponding viruses. See more details in Materials and Methods below.

Identifying Under-Represented Nucleotide Sequences. We report under-represented sequences of size m=3, 4, and 5 nucleotides long in the coding regions of the viruses and in the coding regions of the corresponding hosts in our database. These under-represented sequences are evaluated separately in each of the three reading frames. A common under-represented sequence is a sequence that is under-represented in all three reading frames.

We use two different methods for identifying under-represented sequences in the viruses coding sequences. The first is a random-based approach. In this method, in order to identify under-represented sequences, we compute the p-value of each of the m-nucleotide sequences in each of the three reading frames based on each of our random models. Specifically, we generate 1,000 random variants of each virus in our database based on both of our random models, i.e., for each virus we generated 1,000 dnt samp-based randomizations and 1,000 syn perm-based randomizations.

The random based approach is performed by evaluating the occurrences of each m-size nucleotide sequence, in each reading frame, within each of the 1,000 (syn perm and dnt samp) randomization variants. We then declare a sequence to be under-represented, per reading frame, if the p-value corresponding to syn perm randomization and the p-value corresponding to dnt samp randomization are both less than or equal to 0.05. If, for example, the p-value of a sequence is larger [smaller] than 0.05 relative to the dnt samp [syn perm] randomization model, then its low abundance is assumed to be explained by the dinucleotide frequency and is thus not declared as an under-represented sequence.

In the specific case of m=3, an under-represented sequence that is common to all three reading frames is identified using only the dnt samp randomization, since, per definition, a sym_perm randomization results in no under-represented sequences in the first reading frame in the case of sequences of size m=3.

The second approach, referred to as host-based approach, evaluates the p-values based on the corresponding host sequence rather than a randomization model. The rational here is to identify sequences that are under-represented relative to the corresponding hosts and not necessarily relative to a background model that preserves canonical translation features. Specifically, for each virus the corresponding hosts (i.e., the hosts that are infected by the virus) serve as “random models”. For each corresponding host, its coding regions are divided into consecutive segments of nucleotides with a size equal to the virus coding regions size. Each segment serves as a “random variant” of the virus. Then, for each reading frame we compute the p-value of each of the m-size nucleotide sequence relative to each of the corresponding host. A sequence is declared under-represented, per reading frame, if its p-value is less than or equal to 0.05 relative to all the corresponding hosts. In order to compare between host-based and random-based results, cases with less than 1000 “random variants” in the host were excluded. Since the genome size of most of the dsDNA viruses are relatively large, host-based results for most dsDNA viruses are not available.

The set of under-represented sequences identified in a random variant of a virus (i.e., a random viral genome) was determined as follows. Given a virus, for each random model we randomly picked one (out of 1000) random variant of the virus (that was generated by the random model) and computed the p-value of each m-size nucleotide sequence within this random variant relative to the other 999 random variants. A sequence was declared under-represented if the p-values corresponding to both randomization models are both less than or equal to 0.05. This procedure was used to estimate the false discovery rate of our method, and to compare detection of under-represented sequences between the original genome and a random variant of the genome.

Due to the hosts genome size (which is on average larger than the corresponding virus' size), a direct analysis of under-represented sequences in the hosts, as was described above for the viruses, is infeasible. Our approach for evaluating under-represented sequences in the hosts is based on uniformly “sampling” the host coding regions and evaluating under-represented sequences only in these samples. Specifically, for each host and a corresponding virus pair we used 20 samples, uniformly distributed over the host genome, where each sample's nucleotide size is equal to the total size of the corresponding virus coding sequences. Since the host genomes are “large enough”, it is statistically expected that these sampled under-represented sequences will represent well the actual under-represented sequences in the host.

A sequence is then declared under-represented (per reading frame) in the host given a corresponding virus if it is under-represented (per reading frame) in at least 10 of the 20 samples. The set of these under-represented sequences is denoted as the “sampled majority under-represented set”. This is done separately for each (‘host’, ‘virus’) pair, where ‘virus’ is one of the viruses corresponding to the ‘host’.

The reported host's under-represented sequence set is then determined as follows:
1. For each host, identify its corresponding viruses. Let K denote the number of corresponding viruses.
2. For each corresponding virus, determine the host under-represented sequence set given that virus as described above (i.e., the sequences that are under-represented, per reading frame, in at least 10 of the 20 samples). This is the sampled majority under-represented set of the host given a corresponding virus. Let sᵢ^{j}, iϵ1, K, jϵ1,2,3 denote the set of under-represented sequence in reading frame j given the i'th corresponding virus.
3. The set of under-represented sequences in reading frame j of the host is then defined as s^{j}:=∩_{i=K} sᵢ^{j}, i.e., the intersection over all corresponding viruses of the sets sᵢ^{j}, iϵ1, . . . K.
   Thus, the set of under-represented sequences identified in a host is in some sense relative to its corresponding set of viruses.

To determine under-represented nucleotide sequences that are shared in both host and corresponding virus or that are unique to the host or the corresponding virus, let v denote the set of under-represented sequences in a virus and h denote the sampled majority under-represented set (that is the set of under-represented sequences identified in a host based on a single corresponding virus). Then, for each pair of host-virus (i.e., a host and one of its corresponding virus) we define by (here x denote all the sequences of size of x that are not in x):
(v,h):=v∩h the set of sequences that are under-represented in both host and virus.
(nv,h):=v ∩h the set of sequences that are under-represented in the host but not in the virus.
(v,nh):=v∩h the set of sequences that are under-represented in the virus but not in the host.
(nv,nh):=v ∩h the set of sequences that are not under-represented in the virus nor in the host. This set contains the majority of the sequences (from obvious reasons) and thus will not be analyzed.

Subsets of Host and Viruses. In many cases we present results for subsets of hosts and subsets of viruses. The host subsets are denoted by the pair “H:V”, indicating all hosts of domain H that are infected by viruses of type V (H defines the first two letters of the host domain). For example, Ve:dsDNA denotes all hosts of the domain vertebrate that are infected by viruses of type dsDNA. The virus subsets are denoted by the pair V:H, indicating all viruses of type V that infect hosts of domain H. For example, ssRNA:Pl denotes all the ssRNA viruses that infect hosts of domain plants. The subsets analyzed in the paper, based on the number of hosts or viruses in the subset, are listed in Table 3.

TABLE 3
The different subsets of viruses and hosts used in the analysis.

| Host Domain | Virus Type | Number of viruses | Number of hosts |
|---|---|---|---|
| Plants (Pl) | ssRNA | 141 | 21 |
| Plants (Pl) | ssDNA | 124 | 12 |
| Metazoa (Me) | ssRNA | 57 | 15 |
| Vertebrate (Ve) | ssRNA | 329 | 37 |
| Vertebrate (Ve) | dsDNA | 227 | 33 |
| Vertebrate (Ve) | ssDNA | 131 | 17 |
| Bacteria (Ba) | dsDNA | 1424 | 356 |
| Fungi (Fu) | dsRNA | 18 | 4 |

Classification of Viral Genes into Functional Groups. The viral coding regions were classified into five mutually exclusive functional groups: surface genes, structural genes, enzyme genes, hypothetical (i.e., genes that not certainly encode a protein), and unclassified (i.e., genes with unknown functionality). The group of each gene was determined by analyzing the annotations in the related Fasta file headers according to a list of functional semantic keywords collected from a comprehensive literature survey. In addition, to improve the precision of our classification we used basic semantic relations between the keywords. For example: annotation containing an enzyme/surface keyword was classified as enzyme even if keywords from other structural groups appeared; annotations containing hypothetical keywords and keywords from other groups were assigned to the corresponding group (not to hypothetical group). Finally, the classification results were manually reviewed. The semantic keywords used for the classification of the coding regions into functional groups are:

Surface_keywords: recognition, receptor, surface, membrane, spike, glycoprotein, envelope, env, hn, hemagglutinin, fusion protein.
Structural keywords: capsid, coat, core, matrix, structural protein, virion protein, attachment protein, capsomer, tegument, nucleoprotein, packaging protein, gag, pol, tail protein, head protein, ‘neck protein, portal protein, binding protein, tape measure protein, head-tail joining protein.
Enzymes keywords: enzyme names ending with the “ase” suffix.
Hypothetical proteins keywords: hypothetical protein, putative protein, predicted protein.

For each virus in the database, we divided its genome into the five gene groups (sets) defined above. Each gene set contains all the virus genes of the same functional group. For example, the surface gene set in a virus contains all the genes that encode surface proteins in the virus's genome. Note that a set might be empty in a virus if no genes of the corresponding functional group exist in the virus. The analysis of under-represented sequences was then performed separately in each of the five gene sets for each of the viruses in the database.

Sampled dsDNA Viruses. The genomes of dsDNA viruses are on average longer than the average RNA virus size. In our database, the median nucleotides length (over all virus's coding sequences) is 36,504 in dsDNA viruses, 11,649 in dsRNA viruses, and 9411 in ssRNA viruses. Thus, we should statistically (and not necessarily biologically) expect to find on average more under-represented sequences in dsDNA viruses than in RNA viruses.

To verify this, we have evaluated under-represented sequences in sampled version of all dsDNA viruses in our database. That is, we sampled the virus coding regions and generated a shorter version of the virus (which is referred to as the “sampled virus”). This sampled version was analyzed using the same pipeline as the regular viruses in order to evaluate its under-represented sequences.

The sample size was determined as follows. For each dsDNA virus in our database, we set its sampled size to be the average over all the nucleotide sizes of RNA virus (both single-stranded and double-stranded) that infect the same hosts infected by that dsDNA virus. It was noted that most of the sampled dsDNA viruses have either a size of about 3500 nucleotides, or a size of about 12,000 nucleotides. The former corresponds to dsDNA viruses infecting 14 unique bacteria hosts, and the latter to 23 unique hosts, 21 of which are vertebrate, one metazoa (taxid=7158) and one bacterium (taxid=317).

Zika Virus (ZIKV). ZIKV is a single-stranded, positive-sense RNA virus, which is a member of the family Flaviviridae; it is spread by daytime active Aedes mosquitoes, such as A. aegypti and A. albopictus. The whole ZIKV genome is translated as a polyprotein, which is processed co-translationally and post-translationally by host and viral proteases; the polyprotein contains structural proteins that form the virus particle, and nonstructural proteins that perform various viral functions such as polyprotein processing, genome replication, and manipulation of host responses for viral infection. The largest non-structural protein of ZIKV is the NS5 protein that consists of methyltransferase (MTase) and RNA-dependent RNA polymerase (RdRp) domains, separated by a short linker; the RdRp is essential for viral replication, while the MTase is involved in the viral mRNA capping process, affecting viral detection by the innate immune mechanisms of the host. There is currently no effective vaccine in use to protect from ZIKV. Vaccine development is also complicated by high immune cross-reactivity between Zika and Dengue viruses, which are usually endemic in areas affected by ZIKV.

Preparation of Synthetic Attenuated ZIKV Vaccine based on Under-Represented Oligos. The genome of a Thai-strain ZIKV from an infectious-clone plasmid was evaluated to uncover under-represented sequences. First, the two randomized models (dinucleotides and synonymous codons) were used on the ZIKV coding sequence to identify short sequences that are under-represented. Next, oligos of five nucleotides (5-mers) that identified by both models and showed significant p-values were selected and ranked according to their significance level (see the list of oligos detected in Table 4). Following, the sequence of the Thai strain ZIKV NS5 protein was systematically scanned at the nucleotide level (according to the significance in the relevant frame) in order to identify locations that can be modified with each 5-mer, but without affecting the amino acid sequence of the protein. Specifically, we were able to identify and introduce 29 synonymous codon changes in the first reading frame, and 70 synonymous codon changes in the second reading frame.

The modified NS5 sequence (hereafter named UR99, SEQ ID NO: 3) was later synthesized as plasmid DNA, amplified by PCR, and used in order to build ZIKV-UR99 strain by Gibson assembly. The first-passage stock virus was produced using Vero cells.

Synthetic strain preparation: The infectious-clone plasmid of the Thai-strain ZIKV was constructed from PCR products of viral cDNA. The transfection of the plasmid into mammalian cells generated infectious virus with replication kinetics similar to those of the original virus. The sequence of the infectious-clone plasmid was indeed verified. The viral sequence from this infectious-clone plasmid was evaluated to uncover under-represented sequences as discussed above.

Cell lines: BHK21 with rtTA3 was used to generate virus from assembled DNA. The supernatant from the transfected BHK21 was then used to infect Vero cells in order to prepare the virus stock for subsequent experiments. Replication kinetics of the WT virus and the UR99 virus were characterized in Vero cells with MOI=0.01. The infectious titer was quantitated with Vero cells using immunostaining against E protein by 4G2 monoclonal antibodies.

Animals: 45 male and female AG129 mice produced by an in-house colony were used. Groups of animals of both genders were randomly assigned to experimental groups and individually marked with ear tags. Animals were challenged with Malaysian ZIKV, ZIKV WT synthetic, UR99, or vehicle. Serum was collected from all mice 14 dpi for assessment of neutralizing antibodies via PRNT assay. Mice were monitored for mortality and disease signs daily. Individual weights were recorded daily throughout the course of the study.

Virus: WT Zika virus (Malaysian strain, P6-740) was prepared by two passages in Vero cells. A challenge dose of about 100 CCID50 was administered via s.c. injection in a volume of 0.1 ml.

Quantification of neutralizing antibody: Neutralizing antibody was quantified using a 50% plaque reduction neutralization titer (PRNT50) assay. Serum samples were heat inactivated at 56° C. for 30 minutes in a water bath. One half serial dilution, starting at a 1/10 dilution, of test sera was made. Dilutions were then mixed 1:1 with an appropriate titer of ZIKV in MEM containing 2% fetal bovine serum (FBS) and incubated at 4° C. overnight. The virus-serum mixture was then added to individual wells of a 12-well tissue culture plate with Vero76 cells (4e5 cells/well). Viral adsorption proceeded for one hour at 37° C. and 5% CO2, followed by addition of 1.7% (4000 cps) methylcellulose overlay medium containing 10% FBS to each well. Plates were incubated for four days, and then stained with crystal violet (with 1% (wt/vol) crystal violet in 10% (vol/vol) ethanol) for 20 minutes. The reciprocal of the dilution of test serum that resulted in larger than 50% reduction in average plaques from virus control was recorded as the PRNT50 value.

Overlap between Transcription Factors Binding Sites and Under-Represented Nucleotide Sequences in Viruses. The Jaspar database is an open-access database of curated, non-redundant transcription factor binding profiles. To evaluate the overlap between under-represented sequences in viruses and transcription factor binding sites (TFBS), we first downloaded all the transcription factors binding sites in Jaspar for the following groups: vertebrate (507 TFBS), fungi (176), plants (489) and insects (133). For each set of viruses corresponding to the same host, we selected the most abundant sequences among the common under-represented sequences in the viruses in that set. This set thus contains the most abundant common under-represented sequences in all viruses corresponding to the same host. We then evaluated the overlap between this set and the TFBSs downloaded from Jaspar, for each host domain.

Overlap between Restriction Sites and Under-Represented Sequences of Random Variants of Viruses. In order to show that the selection against short palindromic sequences in viruses cannot be explained by basic translation features such as amino-acids content and order, codon usage bias and dinucleotide distributions, we evaluated the overlap between restriction sites and common under-represented sequences of random variants of viruses as opposed to the original viruses (that was reported in the main paper). Comparing these results to FIG. 5 suggests that indeed the selection against palindromes that are binding sites cannot be explained by the canonical translation features.

Under-Represented Sequences in Zika Virus. Table 4 lists the under-represented sequences of size m=5 nucleotides that were identified in the Zika virus in each reading frame. It should be noticed that these under-represented sequences differ between the three reading frames.

TABLE 4
List of 5-mer oligoes that are under-represented
in the Zika virus, identified using both random models,
and their corresponding p-values (shown separately
for each random model).

| Frame 1 | | Frame 2 | | Frame 3 | |
|---|---|---|---|---|---|
| | p-value | | p-value | | p-value |
| | syn_perm, | | syn_perm, | | syn_perm, |
| Seq. | dnt_samp | Seq. | dnt_samp | Seq. | dnt_samp |
| AGGGG | 1e-3, 2e-3 | TCTTT | 1.5e-3, 1e-2 | CGAGA | <1e-3, 1.4e-2 |
| GATAT | 1.4e-3, <1e-3 | GGGTG | 9e-3, 1.7e-3 | GATCG | 1e-3, 3.6e-2 |
| AAACA | 8e-3, 5e-3 | CACTT | 1.4e-2, 1.2e-2 | TATGT | 3e-3, 4.1e-2 |
| CTCGA | 1e-3, 4.7e-2 | GGACT | 2.8e-2, 8e-3 | GTGGC | 1.5e-2, 1.3e-2 |
| GGGCT | 5e-3, 1.1e-2 | GGCTC | 2e-2, 2.9e-2 | CAAAA | 4.4e-2, 7e-3 |
| AGCCT | 4.7e-2, 1.8e-2 | TGGTT | 2.1e-2, 3.7e-2 | CAGGG | 3.4e-2, 1e-2 |
| | | | | TATTG | 4.3e-2, 2.8e-2 |

Under-Represented Sequences in Sampled dsDNA Viruses. The average number (over all corresponding viruses) of under-represented sequences found in dsDNA viruses was compared to the number found in RNA viruses, and in the sampled dsDNA viruses compared to the number found in RNA viruses, in all three reading frames. The differences in the average number of URs detected decreases when comparing two similar sequence sizes.

For example, in the first reading frame, 9.02 under-represented sequences of size m=3 were found on average in the sampled dsDNA viruses, 23.3 sequences were found on average in dsDNA viruses, and 6.12 sequences were found on average in RNA viruses. For m=4, 6.37 sequences were found on average in the sampled dsDNA viruses, 29.4 sequences were found on average in dsDNA viruses, and 6.44 sequences were found on average in RNA viruses. For m=5, 8.53 sequences were found on average in the sampled dsDNA viruses, 78 sequences were found on average in dsDNA viruses, and 9.05 sequences were found on average in RNA viruses. The results suggest that the number of under-represented sequences identified in dsDNA viruses matches their genomic size, when compared to RNA viruses.

Virus Random- and Host-Based Under-Represented Sequences. The random-based under-represented sequences were determined based on random sequences that preserve certain biological characteristics, such as amino-acids content and order, codon usage bias and dinucleotide distributions in the coding regions. On the other hand, host-based under-represented sequences were based on the corresponding hosts, where none of the biological characteristics mentioned above were preserved. However, in this case, the viruses were evaluated based on their corresponding hosts. The average number of common under-represented sequences of size m=3, 4 and 5 that are random- and host-based in the main four virus groups (i.e., ssDNA, dsDNA, ssRNA, and dsRNA) are listed in Table 5. In most cases, the number of common under-represented sequences that are host-based is larger than the corresponding random-based number.

TABLE 5
The average number of common under-represented sequences, per virus
type, evaluated using both random- and host-based approaches detailed
above. In each t cell, the results for m = 3 appear first, followed by the
results for m = 4 and m = 5.

| Virus Type | Host-based | Random-based |
|---|---|---|
| ssRNA | 0.49 (m = 3) | 0.15 |
| | 0.86 (m = 4) | 0.06 |
| | 0.40 (m = 5) | 0.12 |
| ssDNA | 0.17 | 0.11 |
| | 0.32 | 0.02 |
| | 0.04 | <0.01 |
| dsRNA | 0.17 | 0.19 |
| | 0.38 | 0.19 |
| | 0.10 | 0.17 |
| dsDNA | No results are | 2.25 |
| | available | 2.3 |
| | | 4.1 |

For a complete list of under-represented sequences (for each reading frame, and common to all three reading frames) that are host- and random-based, see Zarai et al., 2020, “Evolutionary selection against short nucleotide sequences in viruses and their related hosts”, DNA Res., Vol. 27: 2, herein incorporated by reference in its entirety. In particular, see Supplemental Tables S3 and S4 of Zarai et al., herein incorporated by reference in their entirety, for random-based and host-based sequences, respectively.

As an example, the following are the common under-represented sequences of size m=3 found in the bacteriophage CTX (ssDNA virus):

Random-based:
GGA, GAC, CCC, CGG.
Host-based:
CCA, AAC, CCC, AGC, TGC, CAG, GCG, CGG, CTG.

Note that two hosts in the database are infected by this virus: Vibrio cholerae and Vibrio cholerae O1 biovar El Tor, and the set of host-based sequences above are under-represented relative to both hosts (and in all three reading frames).

The random-based under-represented sequences of size m=3 in viruses were examined for each reading frame. It should be noted that both ATG and TGG were never found under-represented in the first reading frame in any of the viruses. Since ATG and TTG (codons) uniquely encode the amino-acids Methionine and Tryptophan, respectively, it makes sense that these are never under-represented in reading frame one (and thus are never common under-represented sequences).

Table 6 lists the random-based under-represented sequences of size m=3, in each reading frame, in a few viruses. It may be noticed that in general these under-represented sequences differ between the three reading frames and thus, with the exception of Dengue-1, no common under-represented sequences were detected in these viruses.

TABLE 6
Random-based under-represented sequences of size
m = 3 in each reading frame in few selected viruses.

| Virus | Frame 1 | Frame 2 | Frame 3 | Common |
|---|---|---|---|---|
| Zika | GCA, CGA, GGC, | ATA, GGG, CTG | TCA, CGA, TGC, | |
| [ssRNA(+)] | GGT | | GTC, CAG, TGT | |
| PCV-1 | | TGC, TTC, TAG, | TGA, CGT | |
| [ssDNA] | | TAT | | |
| PCV-2 | GCA, TCA, CGA, | TGC, AGT | AAA, GCC, TTG, | |
| | TTA, AAC, TCG | | CCT | |
| Dengue-1 | GCA, GTA, AGC, | ATA, GTA, GCC, | TAA, TCA, CGA, | GGG |
| [ssRNA(+)] | GGC, ATC, CTC, | ATC, GTC, TCG, | TAC, TGC, CAG, | |
| | AAG, GGG, GAT, | GGG, TTG | CGG, GGG, TGT, | |
| | AGT, GGT, CTT | | ATT | |
| Dengue-2 | GCA, GTA, GGC, | GTA, GCC, ATC, | TAA, TCA, CGA, | |
| | GAG, ACT, GGT, | AGG, GGG, CTG, | TAC, TGC, CAG, | |
| | CTT | GTG, TTG | GGG, TGT | |
| Dengue-3 | GCA, CGA, GTA, | GCA, GTA, GCC, | TAA, TCA, CGA, | |
| | ACC, ATC, CAG, | ATC, GTC, GGG, | TGC, CAG, TTG, | |
| | CCG, ACT, AGT, | CTG, TTG, GTT | GAT | |
| | GGT | | | |
| HIV-1 | ACA, CTA, TAC, | CAA, TGA, ATA, | CCA, ATG, TAT | |
| [ssRNA(+)- | TGC, GAG, AGG, | TAC, GTC, TCG, | | |
| RT] | GAT, TCT, GGT, | TTG, TAT, ACT, | | |
| | GTT | AGT | | |
| HIV-2 | CAA, AGC, GAG, | GTA, GGC, GTC, | CAA, CGA, TGA, | |
| | GCT, AGT, GGT, | ACG, CTG, TTG, | ATA, TTA, CAC, | |
| | CTT, GTT | AAT, TAT | AGC, GGT | |

### Example 1: Under-Represented Sequences Appear in Many Virus Types

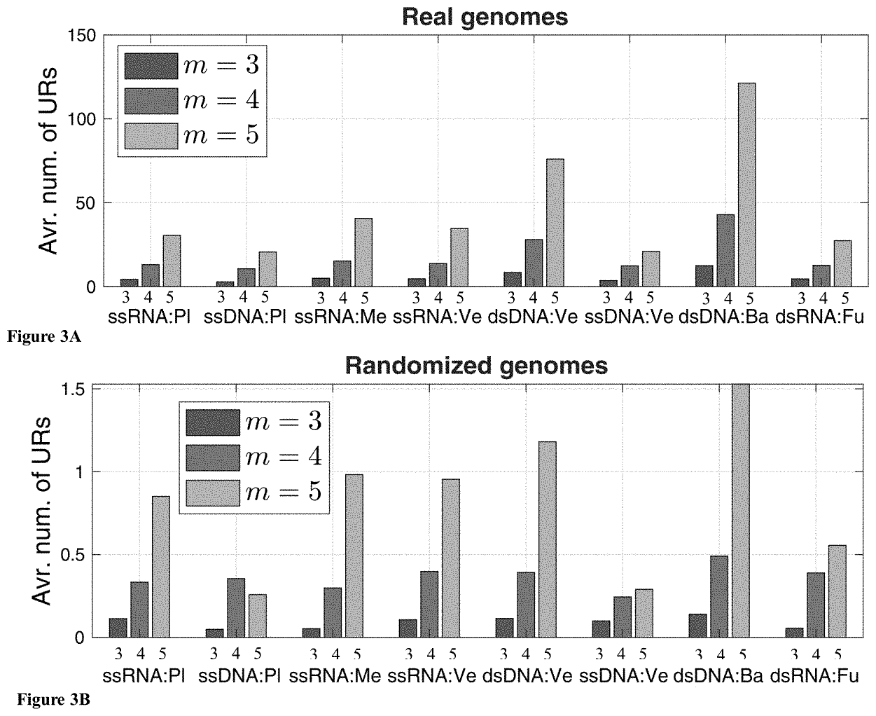

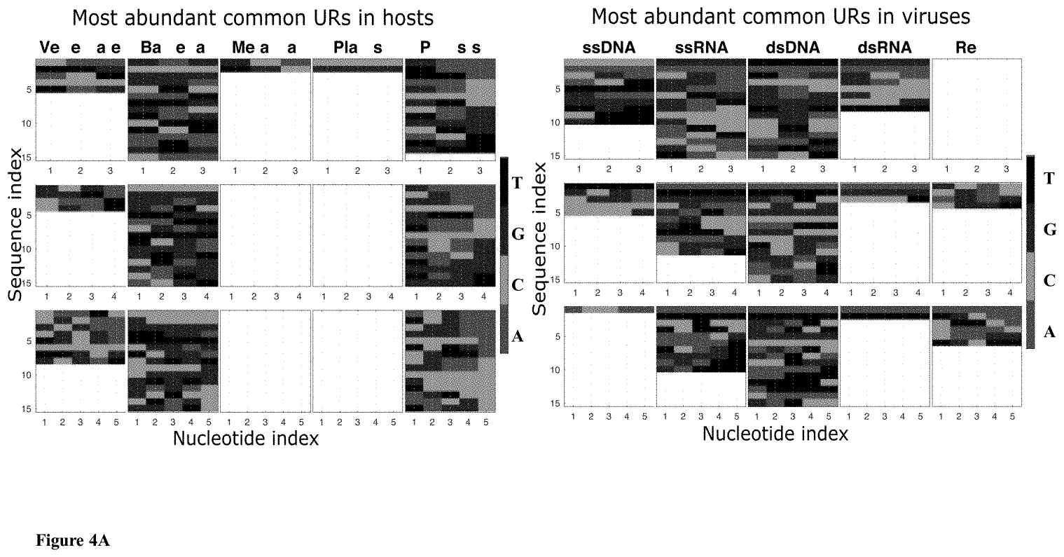

FIGS. 3A-B depict the average number of under-represented sequences of size m=3, 4, and 5 nucleotides, identified in a few subsets of viruses in both the original and random variants of the virus (See Materials and Methods for details about the different subsets, and about random variants of viruses). As shown in these figures, the average number does indeed increase with the sequence size. Also, many under-represented sequences are found in dsDNA viruses that infect bacteria and vertebrate hosts. The average number of under-represented sequences found in the random variants of the viruses is between 1% and 2% of the average number found in the original genome, suggesting a false discovery rate less than 2%.

Since the genome of dsDNA viruses tend to be on average larger than the genome of RNA viruses, we aimed at evaluating if the larger number of under-represented sequences identified can be simply attributed to a better statistical signal due to the larger nucleotide size of these viruses. A sampling analysis that we performed (see Materials and Methods) suggests that the number of under-represented sequences identified in dsDNA viruses matches their genomic size, when compared to RNA viruses.

A complete list of under-represented sequences of sizes m=3, 4, and 5 nucleotides (for each reading frame, and common to all three reading frames) in all viruses in the database is provided in Zarai et al., 2020, “Evolutionary selection against short nucleotide sequences in viruses and their related hosts”, DNA Res., Vol. 27: 2, herein incorporated by reference in its entirety. In particular, see Supplemental Tables S3 and S4 of Zarai et al., herein incorporated by reference in their entirety, for random-based and host-based sequences, respectively.

### Example 2: Evidence of Universal Selection Against Short Homooligonucleotide Mainly within the Viral Coding Regions

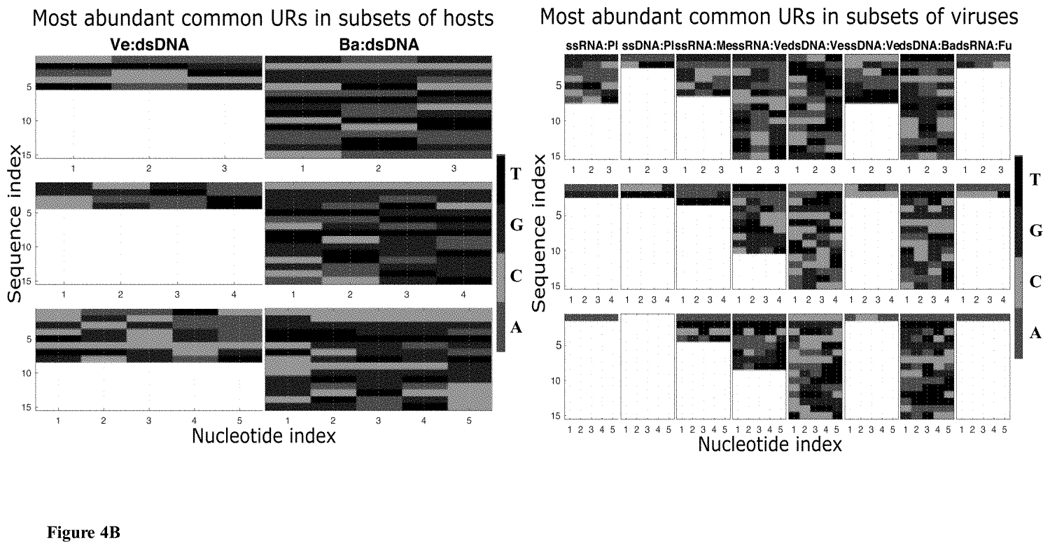

Our analysis suggests that among the most abundant common under-represented nucleotide sequences (i.e., sequences that are under-represented in all three reading frames) are homooligonucleotide repeats, specifically in viruses. These are sequences of the form XX . . . X, where all X contain the same nucleotide. FIG. 4A depicts the most abundant common under-represented sequence in the five host domains (left figure) and in the five main virus groups (right figure).

Note that among these, specifically in viruses, are sequences containing the same nucleotide repeated m=3, 4 or 5 times (i.e., sequences that correspond to the same color repeating m times in the figure). A finer resolution of these common under-represented sequences is provided in FIG. 4B, where we depict these sequences separately for different subsets of hosts (left figure) and subsets of viruses (right figure). See Materials and Methods for more details of the different subsets.

Table 1 lists the six most abundant common under-represented nucleotide sequences of size m=3, 4 and 5 in dsDNA viruses. All homooligonucleotide sequences (shown in bold) are among these most abundant sequences.

TABLE 1

| m = 3 | m = 4 | m = 5 |
|---|---|---|
| TTT (25.5%) | AAAA (25.2%) | AAAAA (29.5%) |
| AAA (18.2%) | TTTT (24.8%) | TTTTT (22.8%) |
| TAG (13.2%) | GATC (24.2%) | GGATC (12.8%) |
| CCC (10.0%) | CGCG (11.1%) | GATCT (11.7%) |
| GAC (8.7%) | GGGG (10.5%) | GGGGG (11.1%) |
| GGG (8.6%) | CCCC (8.2%) | CCCCC (10.5%) |

One possible reason for this general selection against homooligonucleotide (in all three reading frames) in both viruses and hosts is to reduce erroneous frame shifts as the ribosome traverses the mRNA while decoding it codon by codon. A sequence containing a repetition of the same nucleotide in the coding sequence may cause the ribosome to miss the codon boundary, resulting in a frame shift and thus a non-functional and most likely deleterious protein. This must be recognized and degraded by energy-consuming intracellular proteolytic mechanisms. Since translation is the most energetically consuming process in the cell, it is believed that transcripts undergo selection to minimize this energy cost. Selection against sequences of repetitive nucleotides reduces faulty translation, thus minimizing the overall translation cost.

It is possible that this selection against homooligonucleotide repeats is indeed more pronounced in viruses than in hosts since viruses are under much stronger evolutionary selection as they have a larger effective population size and thus a stronger effect of these types of mutations on their fitness. Another possible reason may be related to different host immune evasion mechanisms employed by viruses.

We also evaluated the sequence overlap between common under-represented sequences in viruses and transcription factor binding sites, and again found a general selection against homooligonucleotide repeats. This analysis is reported in the Materials and Methods.

### Example 3: Evidence of Selection Against Short Palindromic Sequences within the Viral Coding Regions

A nucleotide sequence is called palindromic if it is identical to its reverse complement (as opposed to merely reading the same forward and backward). Obviously, palindromic sequences are of even length. Our analysis reveals that 32.5% of all common under-represented sequences of size m=4 nucleotides in viruses are palindromes. Excluding homooligonucleotide repeats this becomes about 51%. It should be noted, that only 6.25% of all possible sequences of size m=4 nucleotides are palindromes. We also evaluated the number of palindromes in random variants of the viruses. These random variants preserve basic transcript features such as amino-acid order and content, codon usage bias and dinucleotide distributions. Only 5.7% of all common under-represented sequences of size m=4 in the random variants of the viruses were found to be palindromes. These findings suggest that indeed the coding regions of viruses are selected against short palindrome sequences.

FIGS. 5A-B depict the percentage of palindromic sequences of size m=4 nucleotides that are common under-represented sequences in subsets of hosts and viruses. It was found that palindromic sequences are selected against only in one subset of hosts: bacterial hosts that are infected by dsDNA viruses. In addition, palindromic sequences were found to be selected against in dsDNA viruses that infect either bacteria (i.e., bacteriophage) or vertebrate hosts.

As depicted in FIG. 5A-B, the sequence GATC is the most abundant palindromic common under-represented sequence in bacteriophages. GATC is a recognition site of different restriction-modification systems, as well as solitary methyltransferase Dam. In addition, methyl-directed Type II DpnI enzyme cleaves methylated GATC sequences. It has been hypothesized that GATC avoidance in bacteria can result from a DNA exchange between strains with different methylation status of GATC site within the process of natural transformation.

FIG. 5C-D depict the total number of occurrences of each palindrome as under-represented sequence in dsDNA viruses that infect bacteria and vertebrate hosts, respectively. In these sub-figures we analyzed under-represented sequences regardless of reading frames. Two cases are shown: the case where the real virus genome is used (shown in blue color), and the case where a randomized variant of the virus genome is used (shown in red color). Note the scale difference in the y-axis between the real and the randomized results. The results in the figures imply that dsDNA viruses undergo selection against short palindrome sequences.

It has been proposed that the principal underlying reason for the apparent avoidance of short palindromes in dsDNA viruses is because they are targets for many restriction-modification systems and possibly for general recombination systems as well. Restriction-modification systems protect bacteria and archaea from attacks by bacteriophages and archaeal viruses. A restriction-modification system specifically recognizes short sites in foreign DNA and cleaves it, while such sites in the host DNA are protected by methylation.

In order to evaluate the hypothesis of palindromes avoidance in viruses due to restriction-modification systems, we downloaded all restriction enzyme patterns from the REBASE database (we used version 811, which contains information for 952 different restriction enzymes) and evaluated the overlap between the common under-represented nucleotide sequences we identified and the restriction sites from REBASE. FIG. 5E depicts the number of exact matches between the most abundant common under-represented palindrome sequences of size m=4 nucleotides in dsDNA viruses and restriction sites. This figure also depicts the corresponding enzyme name and the p-value for each common under-represented sequence. The p-value was computed by evaluating the match between common under-represented sequences of random variants of the viruses and the restriction sites. FIG. 5F depicts the number of restriction sites that are supersets of the most abundant common under-represented palindrome sequences. P-values were computed as in the case of an exact match.

In order to show that the correspondence between selection against short palindromic sequences in viruses and restriction sites cannot be explained by basic coding region features such as amino-acid content and order, codon usage bias and dinucleotide distribution, we also evaluated the overlap between restriction sites and common under-represented sequences of random variants of viruses. This is reported in the Materials and Methods. A complete list of all common under-represented palindromes of size m=4 is provided in Zarai et al., 2020, “Evolutionary selection against short nucleotide sequences in viruses and their related hosts”, DNA Res., Vol. 27: 2, herein incorporated by reference in its entirety. In particular, see Supplemental Tables S6, herein incorporated by reference in its entirety.

### Example 4: Large Numbers of Common Under-Represented Sequences are Found in dsDNA Viruses Infecting Vertebrate or Bacteria Hosts

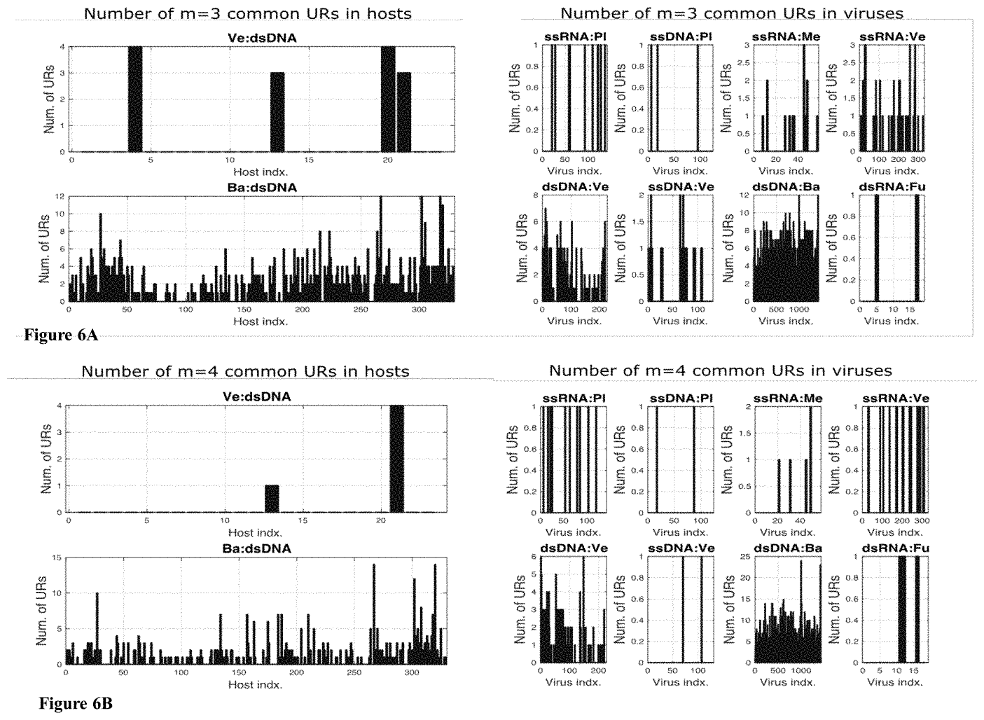

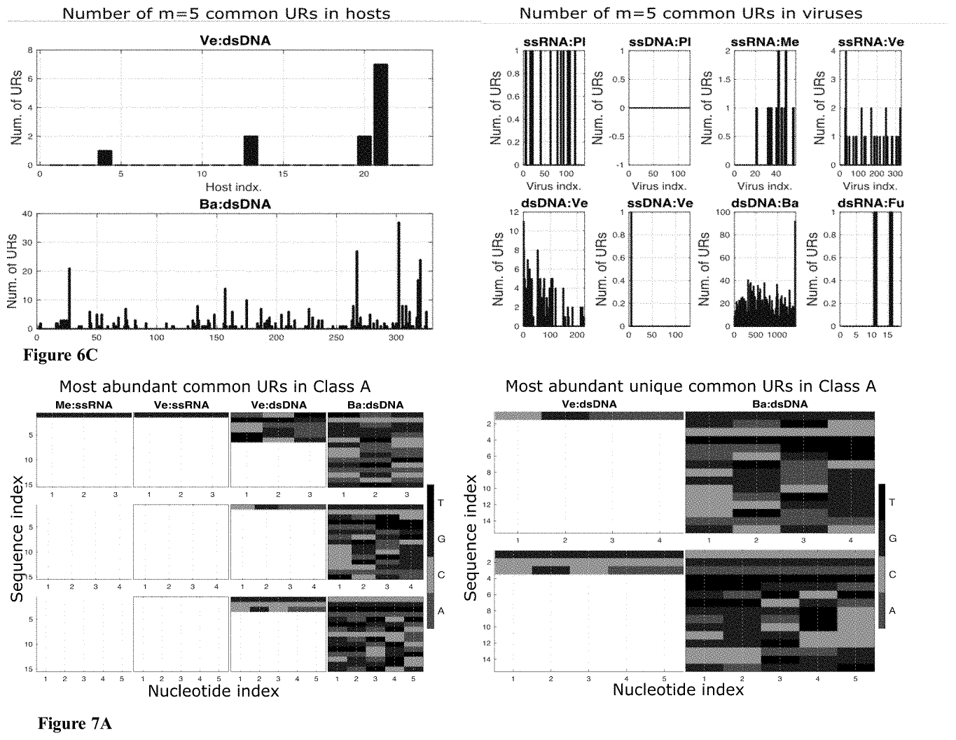

FIGS. 6A-B depict the number of common under-represented nucleotide sequences identified in different subsets of hosts and viruses. Common under-represented sequences were only identified in two subsets of hosts. On the other hand, common under-represented sequences were identified in all eight subsets of viruses.

Our analysis reveals that dsDNA viruses infecting bacteria and vertebrate hosts have the largest number of common under-represented sequences among the different virus subsets. This, as suggested above, seems to be due to the size of dsDNA viruses as compared to ssDNA and RNA viruses. On the other hand, bacteria that are infected by dsDNA viruses have the largest number of common under-represented sequences among the different host subsets. Thus, the stronger selection for under-represented sequences in bacteria may induce stronger selection for under-represented sequences in viruses that utilize this host.

In addition, we evaluated the number of under-represented sequences identified in the real genome of the viruses as compared to the randomized genome of the viruses. This is reported in the Materials and Methods. Indeed, many more sequences are identified as under-represented in the real genome of the virus. On average over all viruses and the three sequence sizes (3, 4, 5), there are about 45 STDs more under-represented sequences in the real genome in comparison to the random genomes, implying that these cannot be explained by basic coding region features, and suggesting possibly new evolutionary forces acting on the viral coding regions.

### Example 5: Many Sequences are Common Under-Represented in Viruses but not in their Related Hosts

We analyzed the correspondence of the under-represented nucleotide sequences between hosts and their related viruses. Specifically, for each pair of a host and a corresponding virus we identified three different classes of sequences:
A. Sequences that are common under-represented in both the host coding regions and in the corresponding virus coding regions.
B. Sequences that are common under-represented in the corresponding virus coding regions but are not common under-represented in the host coding regions.
C. Sequences that are common under-represented in the host coding regions but are not common under-represented in the corresponding virus coding regions.
   It should be noted, that since we analyze each pair of a host and a corresponding virus separately, the set of under-represented sequences in a host above is the sampled majority under-represented set.

For obvious reasons, sequences that are not under-represented in both host and virus coding regions constitute the majority of the sequences and are thus not reported here. A complete list of all under-represented sequences within the three classes above for all hosts and viruses in our database is available in Zarai et al., 2020, “Evolutionary selection against short nucleotide sequences in viruses and their related hosts”, DNA Res., Vol. 27: 2, herein incorporated by reference in its entirety. In particular, see Supplemental Tables S5, herein incorporated by reference in its entirety.

In general, an under-represented sequence of m nucleotides may contain sub-sequences that are themselves under-represented. Thus, it may be interesting to identify unique under-represented sequences, i.e., sequences that do not contain any sub-sequences that are under-represented. For each pair of a host and a corresponding virus, a sequence belonging to one of the three classes above is referred to as a “unique” under-represented sequence if it does not contain any sub sequence that is under-represented in that class. Specifically, a unique common under-represented sequence of size m=4 [m=5] nucleotides doesn't contain any sub sequence of size m=3 [of size m=3 and of size m=4] nucleotides that is common under-represented sequences. A complete list of all unique common under-represented sequences within the three classes above for all hosts and viruses in the database is available in Zarai et al., 2020, “Evolutionary selection against short nucleotide sequences in viruses and their related hosts”, DNA Res., Vol. 27: 2, herein incorporated by reference in its entirety. In particular, see Supplemental Tables S7 herein incorporated by reference in its entirety.

The correspondence of the most abundant under-represented sequences between viruses and their related hosts is depicted in FIGS. 7A-C for different host and virus subsets. Each panel depicts both the most abundant common under-represented sequences (left) and the most abundant unique common under-represented sequences (right), where the panel names correspond to the class names. Our first observation is that many under-represented sequences are indeed unique. For example, comparing the cases of m=4 and m=5 of class A (left, middle and bottom rows, respectively) with the corresponding unique set (right, top and bottom rows, respectively) reveals that the majority of the most abundant sequences are unique. Secondly, homooligonucleotide repeats are among the most abundant sequences in all three classes. In addition, more sequences were identified in class B over the different subsets than in the other two classes. For example, Table 2 lists the most abundant unique sequence of classes B and C in all the different subsets of hosts and viruses. As shown in the table, unique sequences were identified in all subsets for class B, as opposed to class C.

TABLE 2
The most abundant sequence that is unique common under-represented
(of size m = 4 and m = 5) in viruses but not in the corresponding
hosts (top row), and in hosts but not in the corresponding viruses
(bottom row). The numbers in parenthesis indicate the frequency
of occurrences in percentage. X indicates that no corresponding
sequence was identified.

| Pl- | Pl- | Me- | Ve- | Ve- | Ve- | Ba- | Fu- |
|---|---|---|---|---|---|---|---|
| ssRNA | ssDNA | ssRNA | ssRNA | dsDNA | ssDNA | dsDNA | dsRNA |
| AAAA | TTTT | AAAA | TTTT | CCCC | CGGA | GATC | AAAA |
| (6.7) | (1.6) | (4.2) | (1.1) | (11.1) | (0.8) | (22.2) | (1.1) |
| AAAAA | | TTTTT | AAAAA | CCCCC | ACCAA | AAAAA | |
| (7.5) | | (8.3) | (5.3) | (15.3) | (0.8) | (8.2) | |
| X | X | AAAAA | CCCC | GCGA | X | GGGG | X |
| | | (2.1) | (0.9) | (12.1) | | (16) | |
| | | | | CAATC | | TTGGA | |
| | | | | (3.1) | | (9.6) | |

### Example 6: Selection Against Under-Represented Sequences in Viruses Depends on the Protein Function

The viral genome encodes different types of proteins that are necessary for the life cycle of viruses in their respective hosts. These, in general, include surface proteins that interact with the host receptors and enable attachment and entry to the host cell, structural proteins that serve as the building blocks of the virus, and replicating enzymes, such as RNA and DNA polymerase, that are required for the replication of the virus. In addition, many other proteins, some of which are uncharacterized, are diversely involved in different regulatory and accessory functions.

Here, our aim is to refine the analysis of under-represented sequences in viruses by analyzing, separately, different protein groups. To that end, we classified all viral genes into five mutually exclusive functional groups (functional sets): surface, structural, enzymatic, unknown (unclassified genes), and other (hypothetical genes). Specifically, for each virus in the database, we divided its genome into the five gene sets defined above. Each gene set contains all the virus genes of the same functional group. For example, the surface gene set of a virus contains all the genes that encode surface proteins in the virus's genome. A set might be empty for a particular virus if no genes of the corresponding functional group exist in that virus. A list of the total number of sets and genes of each functional group in the database is provided in Zarai et al., 2020, “Evolutionary selection against short nucleotide sequences in viruses and their related hosts”, DNA Res., Vol. 27: 2, herein incorporated by reference in its entirety. In particular, see Supplemental Tables S2, herein incorporated by reference in its entirety. The analysis of under-represented sequences was then performed separately in each of the five gene sets for each of the viruses in the database (see more details in Materials and Methods).

We first analyzed the average number of under-represented sequences identified in each gene set. In order to control for the difference in the average gene size and the number of genes in each set, we randomly selected 1,500, 1,240, 1,450, 3,300 and 2,210 genes from each of the surface, structural, enzymatic, unknown and hypothetical functional groups, respectively. This means that the number of identified under-represented sequences is analyzed over similar region sizes, and the differences between the different sets cannot be explained by the genes' nucleotide size in each set.

FIG. 8A depicts the average number of under-represented sequences (over all three reading frames) identified in each of the gene sets over the (randomly selected) subset of genes. A relatively small number of under-represented sequences was identified in surface genes (that participate in the recognition of the host receptors), as compared to the other gene sets. At least twice as many were identified in many of the enzymatic genes. These proteins interact closely with the host cell machinery, are essential for the viral replication cycle, and thus must employ mechanisms that guarantee their function.

FIG. 8B depicts the most abundant common under-represented sequences within each viral functional group. These differ between the different functional groups, however homooligonucleotide sequences appear among the most abundant common under-represented sequences in all groups.

### Example 7: Under-Represented Sequences Attenuate ZIKV Replication In-Vitro and In-Vivo

We designed an attenuated ZIKV variant based on the under-represented analysis we performed. Such variants are useful in the generating of a live-attenuated vaccine.

Specifically, we introduced synonymous mutations to the NS5 nucleotide sequence, which include under-represented sequences, and named the new variant UR99 (see details in the Methods section, SEQ ID NO: 3). NS5 is an enzymatic protein and not a surface protein.

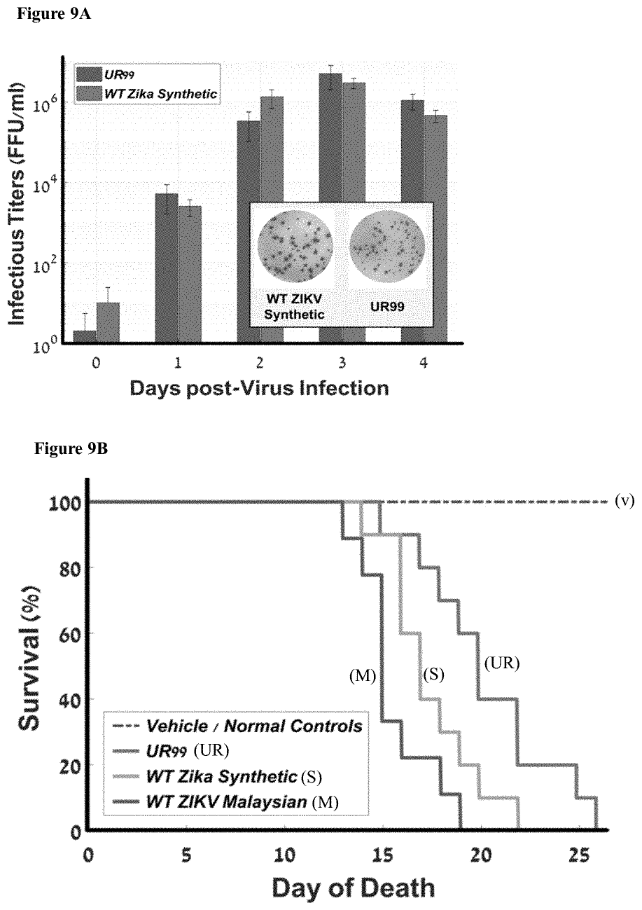

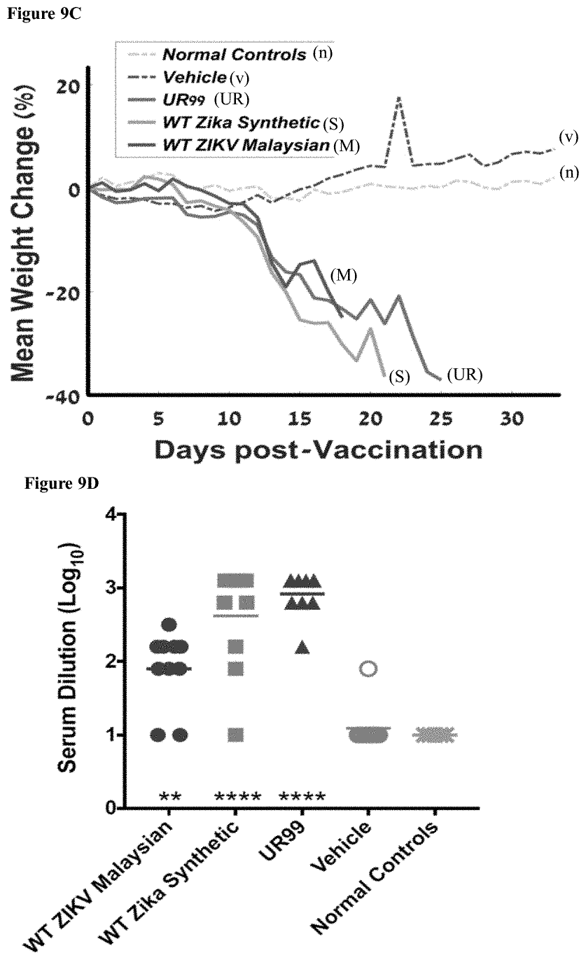

Infection studies in Vero cells demonstrated fractional variant attenuation of the UR99 virus, which was correlative with our model predictions (see foci size in FIG. 9A, right bottom). In addition, infectious virus collected and evaluated from the UR99 variant showed substantial attenuation relative to WT ZIKV (FIG. 9A).

There is evidence that AG129 mice lacking IFN-α/β and IFN-γ (type I and II interferon) receptors, can be valuable for evaluating the efficacy of new vaccines and antiviral treatments for ZIKV. Therefore, as these mice are immune compromised, various strains of ZIKV cause lethal infection and disease, and will typically cause morbidity and mortality. Depending on the strain, severe disease is observed between one and two weeks after virus challenge.

Thus, in order to further test the synthetic vaccine attenuation level in-vivo, AG129 mice were challenged with attenuated ZIKV preparations as well as synthetic WT ZIKV. These inoculations were done in parallel with the original virus grown in cell culture. Infection with the synthetically attenuated ZIKV strains was lethal in all inoculated AG129 mice. However, the mortality curve of mice infected with UR99 was delayed, as compared with that of WT Malaysia and WT synthetic ZIKV (average of 20.4 days in UR99 vs. 15 and 17.5 in WT Malaysia/synthetic ZIKV, respectively; see FIG. 9B). No mortality was observed in unvaccinated controls, and mice vaccinated with vehicle (FIG. 9B).

Weight loss was also observed in all the infected mice (30%-40%; see FIG. 9C). Normal control mice experienced general weight gain throughout the experimental period (FIG. 9C). Weight loss corresponded well with mortality, and mice typically lost substantial weight, requiring humane euthanasia.

Neutralizing antibodies (neutAb) are the primary mediator of protection in vaccine studies in this model. Therefore, serum samples were taken to determine the presence of neutAb in infected mice. The neutAb titer was evaluated in vaccinated mice two weeks after vaccination. Mice vaccinated with synthetic WT or UR99 had significantly (P<0.0001) elevated neutAb titers as compared with vehicle controls (FIG. 9D). As expected, no neutAb was detected in mice vaccinated with vehicle or in normal control groups (FIG. 9D).

The virulence levels of UR99 were lower than the levels of the Malaysian and Synthetic WT strains, thus demonstrating that under-represented sequences can be used in the design of live attenuated ZIKV strains. Accordingly, additional attenuation of this variant (e.g., by introducing similar changes to other ZIKV proteins) would further decreases the lethality of the mice infected by it. Since AG129 mice are very susceptible to ZIKV infection, this mouse model might be too stringent to test these live attenuated vaccine candidates, as human infection is generally subclinical after natural ZIKV infection, hence the attenuated strain might be effective in an immunocompetent model.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A modified viral genome comprising at least one coding sequence comprising at least one mutation, wherein said mutation generates a sequence of 3, 4 or 5 nucleotides that is underrepresented in an unmodified genome of said virus.

2. The modified genome of claim 1, wherein said at least one mutation is a synonymous mutation and does not alter an amino acid sequence encoded by said at least one coding sequence.

3. The modified genome of claim 1, wherein said underrepresented sequence is at least one of:
a. a homooligonucleotide sequence comprising 3, 4 or 5 of the same nucleotide base;
b. a palindromic sequence that is identical to its reverse complement;
c. neither a homooligonucleotide nor a palindromic sequence that is identical to its reverse complement;
d. underrepresented in all three reading frames of said unmodified genome;
e. also underrepresented in a genome of a host organism that is infectable by said virus, optionally wherein said host organism is a bacterium or a vertebrate; and
f. selected from TTT, AAA, TAG, CCC, GAC, GGG, AAAA, TTTT, GATC, CGCG, GGGG, CCCC, AAAAA, TTTTT, GGATC, GATCT, GGGGG, and CCCCC.

4. (canceled)

5. (canceled)

6. (canceled)

7. (canceled)

8. (canceled)

9. (canceled)

10. (canceled)

11. (canceled)

12. The modified genome of claim 1, wherein said virus is ZIKA virus.

13. (canceled)

14. The modified genome of claim 1, wherein said virus is:
a. a double stranded DNA virus that infects bacteria and said underrepresented sequence is a palindromic sequence selected from TTAA, ATAT, CTAG, TGCA, ACGT, CATG, TATA, GTAC, AGCT, GCGC, TCGA, CCGG, AATT, GGCC, CGCG, and GATC;
b. a double stranded DNA virus that infects vertebrates and said underrepresented sequence is a palindromic sequence selected from TTAA, TGCA, TCGA, TATA, GTAC, GGCC, GCGC, GATC, CTAG, CGCG, CCGG, CATG, ATAT, AGCT, ACGT and AATT; or
c. a double stranded DNA virus that infects vertebrates and said underrepresented sequence is a palindromic sequence selected from GATC, GTAC, TGCA, ACGT, GGCC, TCGA, and AATT.

15. The modified genome of claim 1, wherein at least one of:
a said mutated coding sequence encodes a protein identical to an unmutated coding sequence, and wherein a mutated coding sequence comprises every mutation that increases the number of underrepresented sequences without altering the encodes protein sequence;
b. said mutated coding sequence comprises at least 90 mutations;
c. said coding sequence encodes a protein that is not a surface protein; and
d. said coding sequence encodes a surface protein and said underrepresented sequence is a sequence selected from ACT, CTT, GAC, CCT, AAA, AGG, AAT, AGT, TGA, ACC, GTC, TTC, TAG, GGG, GTG, CTAC, CCCC, ACCT, TTTT, CCCCC, TTGCC, and CTTGC.

16. (canceled)

17. (canceled)

18. (canceled)

19. The modified genome of claim 1, wherein said coding sequence encodes at least one of:
a. a structural protein and said underrepresented sequence is a sequence selected from TTT, AAA, GAG, GGG, GGA, CCC, GCT, GCC, CGA, GTC, AGG, CTC, TGT, GAC, AAT, TTTT, AAAA, GATC, GGGG, GGCT, AATT, CGCG, AGCT, GCTT, CCCC, GGAG, GTAC, AAAT, AGCC, TCAG, AAAAA, TTTTT, GGATC, GATCA, CCTGG, AAATT, CGCGC, TTTTC, AATTT, CTTCA, CCCCC, AGATC, and AGCTC;
b. an enzymatic protein and said underrepresented sequence is a sequence selected from TTT, AAA, GAG, GGA, CGC, TGT, GGG, AAT, GCT, TAG, AGG, CGA, GTC, GAC, CCC, AAAA, TTTT, GATC, CGCG, AATT, AGCT, GGCT, TCGA, GGGG, CCCC, GGCC, GCGC, GGAG, GTAC, TTGG, AAAAA, TTTTT, GATCT, AGATC, GGATC, GATCA, CCTGG, AAATT, AATTT, CCCCC, CGATC, TTCGA, CTTGG, TTTTC, and AGCTT;
c. a protein of unknown or uncharacterized function and said underrepresented sequence is a sequence selected from TTT, AAA, TAG, CCC, TGT, CGC, GAC, GGG, AAT, ACA, CTC, GAG, GTC, GCT, GGA, AAAA, TTTT, GATC, CCCC, GGCC, CGCG, GGGG, AATT, AGCT, CCGG, GGCT, GCGC, GTAC, AGCC, TCGA, AAAAA, TTTTT, AGATC, GATTCT, GATCA, GGATC, CCCCC, AATTT, CCTGG, GGGGG, AAAAT, AAATT, TGGCT, AGCTT, and ATTTT; and
d. a protein of known or characterized function that is not a surface, structural or enzymatic protein and said underrepresented sequence is a sequence selected from TTT, AAA, CCC, GGG, ACT, GAC, TAG, CTC, GGA, CGC, GCG, GTG, GAG, AGT, CGA, AAAA, GATC, TTTT, GGGG, CCCC, AATT, CGCG, GCGC, GGAG, AGCT, GGCT, TCGA, GTAC, GGCC, CTCC, AAAAA, CCCCC, GGGGG, TTTTT, GATCT, AGATC, GGATC, GATCA, CCTGG, CGCGG, CCCCA, AAAAT, CGCGC, GGAGC, and GGCGC.

20. A method of generating a modified genome, the method comprising receiving a sequence of a genome of an organism, selecting at least one coding sequence within said genome sequence and introducing at least one mutation into said at least one coding sequence wherein said mutation generates a sequence of 3, 4 or 5 nucleotides that is underrepresented in an unmodified genome of said organism.

21. The method of claim 20, wherein said at least one mutation is a synonymous mutation and does not alter an amino acid sequence encoded by said at least one coding sequence.

22. The method of claim 20, wherein said underrepresented sequence is a homooligonucleotide sequence comprising 3, 4 or 5 of the same nucleotide base, or a palindromic sequence that is identical to its reverse complement.

23. (canceled)

24. The method of claim 20, wherein said underrepresented sequence is at least one of:
a. neither a homooligonucleotide nor a palindromic sequence that is identical to its reverse complement;
b. underrepresented in all three reading frames of said unmodified genome;
c. also underrepresented in a genome of a host organism that is infectable by said virus, optionally wherein said host organism is selected from a bacterium and a vertebrate; and
d. is selected from TTT, AAA, TAG, CCC, GAC, GGG, AAAA, TTTT, GATC, CGCG, GGGG, CCCC, AAAAA, TTTTT, GGATC, GATCT, GGGGG, and CCCCC.

25. (canceled)

26. (canceled)

27. (canceled)

28. (canceled)

29. The method of claim 20, wherein said virus is ZIKA virus.

30. (canceled)

31. The method of claim 20, wherein said virus is:
a. a double stranded DNA virus that infects bacteria and said underrepresented sequence is a palindromic sequence selected from TTAA, ATAT, CTAG, TGCA, ACGT, CATG, TATA, GTAC, AGCT, GCGC, TCGA, CCGG, AATT, GGCC, CGCG, and GATC;
b. a double stranded DNA virus that infects vertebrates and said underrepresented sequence is a palindromic sequence selected from TTAA, TGCA, TCGA, TATA, GTAC, GGCC, GCGC, GATC, CTAG, CGCG, CCGG, CATG, ATAT, AGCT, ACGT and AATT; or
c. a double stranded DNA virus that infects vertebrates and said underrepresented sequence is a palindromic sequence selected from GATC, GTAC, TGCA, ACGT, GGCC, TCGA, and AATT.

32. The method of claim 20, wherein said introducing comprises mutating every nucleotide that increases the number of underrepresented sequences within said coding sequence without altering an amino acid sequence of a protein encoded by said coding sequence.

33. The method of claim 20, wherein at least one of:
a. said introducing comprises introducing at least 90 mutations into said at least one coding sequence;
b. said coding sequence encodes a protein that is not a surface protein; and
c. said coding sequence encodes encodes a surface protein and said underrepresented sequence is a sequence selected from ACT, CTT, GAC, CCT, AAA, AGG, AAT, AGT, TGA, ACC, GTC, TTC, TAG, GGG, GTG, CTAC, CCCC, ACCT, TTTT, CCCCC, TTGCC, and CTTGC.

34. (canceled)

35. (canceled)

36. The method of claim 20, wherein said coding sequence encodes at least one of:
a. a structural protein and said underrepresented sequence is a sequence selected from TTT, AAA, GAG, GGG, GGA, CCC, GCT, GCC, CGA, GTC, AGG, CTC, TGT, GAC, AAT, TTTT, AAAA, GATC, GGGG, GGCT, AATT, CGCG, AGCT, GCTT, CCCC, GGAG, GTAC, AAAT, AGCC, TCAG, AAAAA, TTTTT, GGATC, GATCA, CCTGG, AAATT, CGCGC, TTTTC, AATTT, CTTCA, CCCCC, AGATC, and AGCTC;
b. an enzymatic protein and said underrepresented sequence is a sequence selected from TTT, AAA, GAG, GGA, CGC, TGT, GGG, AAT, GCT, TAG, AGG, CGA, GTC, GAC, CCC, AAAA, TTTT, GATC, CGCG, AATT, AGCT, GGCT, TCGA, GGGG, CCCC, GGCC, GCGC, GGAG, GTAC, TTGG, AAAAA, TTTTT, GATCT, AGATC, GGATC, GATCA, CCTGG, AAATT, AATTT, CCCCC, CGATC, TTCGA, CTTGG, TTTTC, and AGCTT;
c. a protein of unknown or uncharacterized function and said underrepresented sequence is a sequence selected from TTT, AAA, TAG, CCC, TGT, CGC, GAC, GGG, AAT, ACA, CTC, GAG, GTC, GCT, GGA, AAAA, TTTT, GATC, CCCC, GGCC, CGCG, GGGG, AATT, AGCT, CCGG, GGCT, GCGC, GTAC, AGCC, TCGA, AAAAA, TTTTT, AGATC, GATTCT, GATCA, GGATC, CCCCC, AATTT, CCTGG, GGGGG, AAAAT, AAATT, TGGCT, AGCTT, and ATTTT; and
d. a protein of known or characterized function that is not a surface, structural or enzymatic protein and said underrepresented sequence is a sequence selected from TTT, AAA, CCC, GGG, ACT, GAC, TAG, CTC, GGA, CGC, GCG, GTG, GAG, AGT, CGA, AAAA, GATC, TTTT, GGGG, CCCC, AATT, CGCG, GCGC, GGAG, AGCT, GGCT, TCGA, GTAC, GGCC, CTCC, AAAAA, CCCCC, GGGGG, TTTTT, GATCT, AGATC, GGATC, GATCA, CCTGG, CGCGG, CCCCA, AAAAT, CGCGC, GGAGC, and GGCGC.

37. (canceled)

38. An attenuated virus comprising a modified genome of claim 1.

39. A vaccine composition comprising the attenuated virus of claim 38.

40. A computer program product for generating a modified genome, comprising a non-transitory computer-readable storage medium having program code embodied thereon, the program code executable by at least one hardware processor to:
a. receive a sequence of a genome of an organism;
b. receive a list of underrepresented sequences of length 3, 4 or 5 nucleotides in said organism;
c. calculate mutations within at least one coding sequence of said genome that generate at least one underrepresented sequence from said list; and
d. provide an output modified genome comprising said at least one coding sequence comprising at least one calculated mutation wherein said at least one calculated mutation does not alter an amino acid sequence of a protein encoded by said at least one coding sequence.

41. The computer program product of claim 40, wherein at least one of:
a. said output modified genome comprises said at least one coding sequence comprising every possible calculated mutation that does not alter an amino acid sequence of a protein encoded by said at least one coding sequence;
b. said list is ranked in order of the extent of underrepresentation in the genome; and
c. said list is ranked in order of the extent of underrepresentation in the genome and said output modified genome comprises the 5 most highly underrepresented sequences that could be generated by a synonymous mutation.

42. (canceled)

43. (canceled)


SEQUENCE LISTING (3 sequences)

SEQ ID NO: 1 - protein, 903 aa, Zika virus
GGGTGETLGEKWKARLNQMSALEFYSYKKSGITEVCREEARRALKDGVATGGHAVSRGSAKLRWLVERGYLQPHGKVIDLGCGRGGWSYYAATIRKVQEVKGYTKGGPGHEEPMLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLVNGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREYHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRPEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 2 - DNA, 2712 bp, Zika virus
  source 1..2712: mol_type = genomic DNA
gggggtggaacaggagagaccctgggagagaaatggaaggcccgcctgaaccagatgtcggccctggagttctactcctacaaaaagtcaggcatcaccgaggtgtgcagagaagaagcccgccgcgccctcaaggacggtgtggcaacgggaggccatgctgtgtcccgaggaagtgcaaagctgagatggttggtggagcggggatacctgcagccccatggaaaggtcattgatcttggatgtggcagagggggctggagctactacgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctatgaggcccctacacaagggtcagcgtcctctctggtaaacggggttgtcaggctcctgtcaaaaccctgggatgtggtgactggagtcacaggaatagctatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagtaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacattcaccaacctagtggtgcagctcattcggaatatggaggctgaggaagttttggagatgcaagacttgtggctgctgcggaggccagagaaagtgaccaactggttgcagagcaacggatgggataggctcaaacgaatggcagtcagtggagatgattgcgttgtgaagccaattgatgataggttcgcacatgccctcaggttcttgaatgatatgggaaaagttaggaaggacacacaagagtggaaaccctcaacaggatgggacaactgggaagaagttccgttttgctcccaccatttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaaatacatggactacctatccacccaagtgcgctacttgggtgaagaagggtctacacctggagtgctataa

SEQ ID NO: 3 - DNA, 2712 bp, synthetic construct
  misc_feature 1..2712: note = Synthetic
  source 1..2712: mol_type = other DNA
gggggtgggactggagagacacttggagagaaatggaaggcccggctcaaccagatgtcggccctcgagttctactcctacaaaaagtcaggcatcaccgaggtgtgcagagaagaagcccgccgcgcacttaaggacggggtggcaacgggaggccatgctgtgtccaggggaagtgcaaagctgagatggctcgtggagaggggatacctgcagccccatggaaaggtcattgatcttggatgtggcagggggggctggagctactacgccgccaccatccgcaaagttcaagaagtgaaaggatacacaaaaggaggccctggtcatgaagaacccatgttggttcaaagctatgggtggaacatagtccggctcaagagtggggtggacgtctttcatatggcggctgagccgtgtgacacacttctgtgtgatataggtgagtcatcatctagtcctgaagtggaagaagcacggactctcagggtgctctccatggttggggattggctcgaaaaaagaccaggagccttttgtataaaagtgttgtgcccatacaccagcactatgatggaaaccctcgagcggctccagcgtaggtatgggggagggctggtcagggtgccactttcccgcaactctacacatgagatgtactgggtgtctggagcgaaaagcaacaccataaaaagtgtgtccaccacgagccagctcctcttggggcgcatggacgggcccaggaggccagtgaaatatgaggaggatgtgaatctcggctctgggactcgggctgtggttagctgcgctgaagctcccaacatgaagatcattggtaaccgcattgagaggatccgcagtgagcacgcggaaacgtggttctttgacgagaaccacccatataggacttgggcttaccatggaagctatgaggcccctacacaagggtcagcgtccagcctggtaaacggggtggtcaggctcctgtcaaaaccctgggatgtggttactggggtgacaggaatagctatgaccgacaccacaccgtatggtcagcaaagggtgttcaaggaaaaagtggacactagggtgccagacccccaagaagggactcgtcaggttatgagcatggtttcttcctggctctggaaagagctaggcaaacacaaacggccacgggtgtgtaccaaagaagagttcatcaacaaggttcgtagcaatgcagcacttggggcaatctttgaagaggaaaaagagtggaagactgcagtggaagctgtgaacgatccaaggttctgggcacttgtggacaaggaaagagagtaccacctgaggggagagtgccagagctgtgtgtacaacatgatgggaaaaagagaaaagaaacaaggggaatttggaaaggccaagggcagccgcgccatctggtatatgtggctcggggctagatttctcgagttcgaagcacttggattcttgaacgaggatcactggatggggagagagaactcaggaggtggggtggaagggctcgggctacaaaggctcggatatgtcctcgaagagatgagtcgcataccaggaggaaggatgtatgcagatgacactgctggctgggacacccgcatcagcaggtttgatctcgagaatgaagcacttatcaccaaccaaatggagaaagggcacagggcacttgcacttgccataatcaagtacacataccaaaacaaagtggttaaggtccttagaccagctgaaaaagggaagacagttatggatattatttcaagacaagaccaaagggggagcggacaagtggttacttacgcacttaatacattcaccaacctggttgtgcagctcattcggaatatggaggctgaggaagttctcgagatgcaagacttgtggctcctgcggaggccagagaaagtgaccaactggctccagagcaacggatgggataggctcaaacgaatggcagtcagtggagatgattgcgtggttaagccaattgatgataggttcgcacatgcacttaggttcttgaatgatatgggaaaagttaggaaggacacacaagagtggaaaccctcaacaggatgggacaactgggaagaagttccgttttgctcccaccatttcaacaagctccatctcaaggacgggaggtccattgtggttccctgccgccaccaagatgaactgattggccgggcccgggtgtctccaggggcgggatggagcatccgggagactgcttgcctagcaaaatcatatgcgcaaatgtggcagctcctttatttccacagaagggacctccggctcatggccaatgccatttgttcatctgtgccagttgactgggtgccaactgggaggactacctggtcaatccatggaaagggagaatggatgaccactgaagatatgctggttgtgtggaacagggtgtggattgaggagaacgaccacatggaagacaagaccccagttacgaaatggactgatattccctatttgggaaaaagggaagacttgtggtgtggaagcctcatagggcacagaccgcggactacctgggctgagaacattaaaaacacagtcaacatggttcgcaggatcataggtgatgaagaaaaatacatggactacctatccacccaagtgcgctacttgggtgaagaagggtctacacctggggtgctataa